SEQUENCE LISTING (20 sequences)

SEQ ID NO: 1 - protein, 1487 aa, Saccharomyces cerevisiae
MPLLQPSTCFCYPLKLPPLPLTSDSNEFDECARKRLTLDYRTGSAVTLTRSNIFVHGGLTIPLNLPVVNSMQLQKELILFFAKEKNNGSSFRNLNEWISKETFFLDLMSRTWYRVKTSFDQRTEELLKAESSSAKADNDTNEIRTDIKKGKSLESPLKERLFHSLCYLDGCLYIFGGLTVSPQSGYELIATNELWKLDLNTKKWSLLSDDPQIARRFNHTMHVKNENNDNRDTKLIIVGGLNNMDQPVKKIDIYNISQNCWQSETIPKQPMEITTNVNGIPLALSKDQNFSILVENNEANVPALAFYMRSDQIDEYLGKDSSKIKENSPIVALPLLSESQGIRMPSNPALPKKLLNVPYELLAPTGDYFGFNIIIGGFHPNYQSSNFHCFIYDINSGKWSRVATACPDCDINKHRFWRVFVWKSHHQTILLGTKTDDYYSPSVQRFDHLSTFGLPLVNIFNKTIQLPHHKISASSLPIPIENFAKHKDTPLKKVSFTSSATSQFENYIRYIAPPLEMSSIQSVFPPYAMVLGKDALEIYGKPLSDFEFITSEGDSIGIPCYLLRKRWGRYFDMLLSQSYTKVCADYETTDTQSTLIKFSPHSSRNSSKAVRQEGRLSSSGSLDNYFEKNFPIFARTSVSEAQNTQPQVANADAKAPNTPSTSDEPSSSSSSDLYSTPHYQRNNDEEDDEDPVSPKPVSKSNSIYRPIRKTESSSTTSSSNGMIFRVPFKEKAAVTSNTEALLESNLSLQELSRRRSSLMSIPSGELLRSSISEAEHQRRASHPLTSSPLFEDSGTPCGKQLQQLQQHTIQNPHNHLSPRRFSRSARSSISYVSSSSDRRGNSISSRSTSDSFGTPPVLGVLNVPLPPQTREPNEPPPPCPAMSTGSNTRRSNTLTDYMHSNKASPFSSRRSSHIGRRSSTPETENAFSATPRASLDGQMLGKSLKEGSTSQYTQPRMNSFPKANETIQTPTSSNNEWSRQSVTSNTDSFDSLQSNFALELEPLLTPRSLYMPWPTSTVRAFAEFFYTGQVNSKWLLAPVALDLLVMAKIYEIPLLYKLILEVLYSILAKKEESLSLICTSLMETFRTKTLNSYKGDEEKTNTYLTSNDNYQELLKLKVSLENIDNGYYDPDLLRKQSRAQSSSTQESSGSANGEKTATGAGSLETSSTNVPTVFAGGPRDSHNSVGSIGFPNSMNIQGSRRSTSGFSPRVKMKSSLSKEIDPKTFYEEYEPKEGKSFDDNDDQQTNIGSFNLHLFDMNYGSISSSSTNSISSSDLEEKEEQEQLQDLLEIEREDSAEILDARFRNKEDDKVTKDISNDKKRNYLPHEKNNLKAKEGKETRDVREEEEEFDFGLGMLSLNKIKREAKHVDKVDDSVDPLFKSSAFPQSPIRAYGSTTRTSSASGKPFRDNRSFNAFSVLTLENMASANALPPVDYVIKSIYRTTVLVNDIRLMVRCMDCIELSKNLRALKKKTMEDISKLKGISKPSP

SEQ ID NO: 2 - DNA, 4464 bp, Saccharomyces cerevisiae
  source 1..4464: mol_type = genomic DNA
atgcctttattacaaccatccacttgcttctgctaccctttgaagcttccgccattaccactgacttccgattccaacgagtttgatgaatgcgccaggaaaaggttaacgctggactatagaacaggttcagcggtgacgctaacaaggtctaatatatttgtacatggtggtctaaccatcccgttgaacctgccagttgtaaattctatgcaattacaaaaggagcttattctcttttttgcaaaggaaaaaaacaacggtagttcttttaggaatttaaacgagtggataagtaaggagacgtttttcttagacttgatgtctagaacctggtacagagtcaagacctcctttgaccaaaggacagaggaattattaaaggcagagagttctagcgccaaagcagataatgacacaaacgagattagaacggacattaagaaaggtaaatcccttgaatctccgctcaaggagaggctattccattccttatgttatttagatggctgtttatatatatttggtggtctcacagtgtctcctcagagcggatatgagttgattgctaccaatgagttgtggaaactggatttaaacacgaagaagtggtcgcttttgagtgatgaccctcaaatagcgagaaggttcaaccataccatgcatgtaaagaacgaaaataacgataatagagacacgaaattaataattgtcggaggtcttaataacatggatcagccggtcaagaaaatagacatctataacatctcacagaattgctggcaatccgaaaccatacccaaacaaccgatggaaatcactacaaatgtcaatggcataccattagcattatcaaaggatcagaatttttcgattttagttgaaaataatgaggccaacgttccagcgctggcattctatatgagaagtgatcaaatagacgaatatctcggaaaggattcatcaaagataaaggagaattcgcctatagtggcgttgccgctattgtctgaatcgcaaggtattagaatgccctcaaacccggcattaccgaaaaaactattgaatgttccgtatgagctattagcaccgacaggtgactattttggattcaatatcataatcggtgggtttcatccaaattaccaatcctctaactttcattgttttatatacgatattaactccggaaagtggtcacgagtcgctaccgcctgccctgactgcgatatcaataaacatcgattctggagggtatttgtttggaaatcgcatcatcagacgattttgttgggtaccaagactgatgattattattcacccagtgtacaaagattcgaccatctttccacttttggattaccgctggtaaatatttttaacaagacgatccaattaccccatcacaagatctcagcttcgtctttaccaatacccatagaaaattttgcgaagcataaggatactccattaaaaaaggtgagcttcacttcatcagctacctcccaatttgaaaactacattagatatatagctccgcccttggaaatgtcttctatccaatctgtgtttccaccgtatgccatggttctaggtaaagacgctttagagatttacgggaagcctctttctgattttgaatttattactagtgaaggtgactccattggtataccatgctatttgctgaggaaaagatggggccgttatttcgacatgcttttatcccagagttatacaaaagtttgtgctgattatgaaactactgatacgcaatctacactaataaaattttcaccacattcatctagaaacagttctaaagctgtgaggcaagaaggtagactctcttcatcgggttcactggacaattatttcgaaaaaaacttcccaatctttgcgagaacaagtgtttccgaggcccagaacacacaacctcaggtagcgaatgctgatgcgaaagctccaaatacacctagtacgtcagatgaaccaagctcttcctcctcttccgatttatactccactcctcattaccaacgtaataatgatgaagaggatgacgaggacccggtttccccgaagcctgtatcaaaatcaaacagtatctatagacccattagaaagacagaaagttcatcaacaacgagttcttcaaacggtatgattttcagagtacctttcaaggaaaaagcggctgtaacgtcaaacactgaagccctcttggagtcaaacctttcacttcaagaattgagccgaagaagatcatcacttatgagcatcccgtctggtgaacttctaaggtcctctatatctgaagcggaacatcaacgtcgtgcatctcatccactcacttcatcaccgctctttgaagatagtggaaccccttgcggcaaacaactgcaacaactgcaacaacatactatacaaaatcctcataaccatttgtcgccccggaggttttcaagaagtgcaagaagttcaatttcctacgtgagttcttcttccgatagacgtggaaattcaatttctagcaggagcacaagcgatagctttggaactccaccagttttgggcgttttgaatgtaccattacctcctcaaacaagagaacctaatgaaccacctccaccatgtcctgcaatgagtaccggctcaaatacaaggcgaagcaacaccttaacggattatatgcattctaacaaggctagcccgttttcttctcgtagatcgtcgcatatcggtagacgttctagtacaccggaaacggaaaacgcattctctgcaacaccaagagcatccttagatggtcaaatgttaggaaaatctttgaaggaaggttctacatcacagtacacacagccaaggatgaactctttcccaaaggctaatgaaaccatacaaacacccacatcatcaaacaatgaatggagccgtcaatccgttacttcaaatacagatagttttgacagtttgcaatctaattttgcgttagagttggaaccacttttaacaccgaggtcactttatatgccttggccaacctccacagttcgagcatttgcggaatttttttacactgggcaagtaaacagtaaatggcttttggcaccagttgctcttgatttattagtaatggccaagatttatgaaataccattactgtataaattgatccttgaagttttatattcgattctggctaaaaaagaagaaagtttatccttaatatgtacttcgttaatggagacctttcgcaccaaaactctgaactcctataaaggtgatgaagaaaaaacaaatacttatttgacttcaaacgataactatcaggaactgttgaaattaaaagtgtcgctggagaatatcgacaatgggtattatgacccagatttgttacgcaaacagtcaagagcacagtcttcaagcacacaagaaagcagtggtagtgcaaacggcgaaaaaactgcaactggcgctggctctctggagacttcttcaaccaatgtccctacagtatttgctggcggtcccagagatagccacaattcagtaggctccattggttttccaaacagtatgaatatacaaggctcaagaagatcgacatcaggattttctccacgcgttaagatgaaatcgagcttaagcaaggaaatagatcccaaaactttttatgaagagtacgaaccaaaagagggcaaaagttttgatgataacgatgatcaacaaaccaacatcggaagttttaaccttcatttgtttgatatgaattatggttccatcagcagcagtagcactaacagcattagtagcagtgatttagaagagaaagaagaacaagagcaacttcaagatttattggaaatagaaagagaagattctgctgagatcttagacgcaagattcagaaacaaagaagatgataaagtgacgaaggatatatcaaatgacaagaaacgcaattacttaccgcatgaaaaaaataacttgaaagccaaggaaggtaaggaaactagagacgtaagggaggaggaggaagaattcgattttggtttgggaatgctttcgctcaataaaataaaaagagaagccaagcatgtcgataaggtggacgattctgtagaccctttatttaaatcatctgctttccctcaaagtcccattcgagcatacgggtctaccacaagaactagttcggcttctggaaagccattcagggacaatcgttcatttaatgcattttctgttttgacattagaaaatatggcgtctgcaaatgccctacctcccgttgattatgtcataaaatcaatatacagaaccactgtgttagtgaacgatatcagactaatggttcgttgcatggattgcattgaattatctaaaaatttacgagcgctcaagaaaaaaactatggaagatatttcgaaattgaaaggcatctctaaaccgtcaccatag

SEQ ID NO: 3 - protein, 1487 aa, Saccharomyces cerevisiae
MPLLQPSTCFCYPLKLPPLPLTSDSNEFDECARKRLTLDYRTGSAVTLTRSNIFVHGGLTIPLNLPVVNSMQLQKELILFFAKEKNNGSSFRNLNEWISKETFFLDLMSRTWYRVKTSFDQRTEELLKAESSSAKADNDTNEIRTDIKKGKSLESPLKERLFHSLCYLDGCLYIFGGLTVSPQSGYELIATNELWKLDLNTKKWSLLSDDPQIARRFNHTMHVKNENNDNRDTKLIIVGGLNNMDQPVKKIDIYNISQNCWQSETIPKQPMEITTNVNGIPLALSKDQNFSILVENNEANVPALAFYMRSDQIDEYLGKDSSKIKENSPIVALPLLSESQGIRMPSNPALPKKLLNVPYELLAPTGDYFGFNIIIGGFHPNYQSSNFHCFIYDINSGKWSRVATACPDCDINKHRFWRVFVWKSHHQTILLGTKTDDYYSPSVQRFDHLSTFGLPLVNIFNKTIQLPHHKISASSLPIPIENFAKHKDTPLKKVSFTSSATSQFENYIRYIAPPLEMSSIQSVFPPYAMVLGKDALEIYGKPLSDFEFITSEGDSIGIPCYLLRKRWGRYFDMLLSQSYTKVCADYETTDTQSTLIKFSPHSSRNSSKAVRQEGRLSSSGSLDNYFEKNFPIFARTSVSEAQNTQPQVANADAKAPNTPSTSDEPSSSSSSDLYSTPHYQRNNDEEDDEDPVSPKPVSKSNSIYRPIRKTESSSTTSSSNGMISRVPFKEKAAVTSNTEALLESNLSLQELSRRRSSLMSIPSGELLRSSISEAEHQRRASHPLTSSPLFEDSGTPCGKQLQQLQQHTIQNPHNHLSPRRFSRSARSSISYVSSSSDRRGNSISSRSTSDSFGTPPVLGVLNVPLPPQTREPNEPPPPCPAMSTGSNTRRSNTLTDYMHSNKASPFSSRRSSHIGRRSSTPETENAFSATPRASLDGQMLGKSLKEGSTSQYTQPRMNSFPKANETIQTPTSSNNEWSRQSVTSNTDSFDSLQSNFALELEPLLTPRSLYMPWPTSTVRAFAEFFYTGQVNSKWLLAPVALDLLVMAKIYEIPLLYKLILEVLYSILAKKEESLSLICTSLMETFRTKTLNSYKGDEEKTNTYLTSNDNYQELLKLKVSLENIDNGYYDPDLLRKQSRAQSSSTQESSGSANGEKTATGAGSLETSSTNVPTVFAGGPRDSHNSVGSIGFPNSMNIQGSRRSTSGFSPRVKMKSSLSKEIDPKTFYEEYEPKEGKSFDDNDDQQTNIGSFNLHLFDMNYGSISSSSTNSISSSDLEEKEEQEQLQDLLEIEREDSAEILDARFRNKEDDKVTKDISNDKKRNYLPHEKNNLKAKEGKETRDVREEEEEFDFGLGMLSLNKIKREAKHVDKVDDSVDPLFKSSAFPQSPIRAYGSTTRTSSASGKPFRDNRSFNAFSVLTLENMASANALPPVDYVIKSIYRTTVLVNDIRLMVRCMDCIELSKNLRALKKKTMEDISKLKGISKPSP

SEQ ID NO: 4 - DNA, 4464 bp, Saccharomyces cerevisiae
  source 1..4464: mol_type = genomic DNA
atgcctttattacaaccatccacttgcttctgctaccctttgaagcttccgccattaccactgacttccgattccaacgagtttgatgaatgcgccaggaaaaggttaacgctggactatagaacaggttcagcggtgacgctaacaaggtctaatatatttgtacatggtggtctaaccatcccgttgaacctgccagttgtaaattctatgcaattacaaaaggagcttattctcttttttgcaaaggaaaaaaacaacggtagttcttttaggaatttaaacgagtggataagtaaggagacgtttttcttagacttgatgtctagaacctggtacagagtcaagacctcctttgaccaaaggacagaggaattattaaaggcagagagttctagcgccaaagcagataatgacacaaacgagattagaacggacattaagaaaggtaaatcccttgaatctccgctcaaggagaggctattccattccttatgttatttagatggctgtttatatatatttggtggtctcacagtgtctcctcagagcggatatgagttgattgctaccaatgagttgtggaaactggatttaaacacgaagaagtggtcgcttttgagtgatgaccctcaaatagcgagaaggttcaaccataccatgcatgtaaagaacgaaaataacgataatagagacacgaaattaataattgtcggaggtcttaataacatggatcagccggtcaagaaaatagacatctataacatctcacagaattgctggcaatccgaaaccatacccaaacaaccgatggaaatcactacaaatgtcaatggcataccattagcattatcaaaggatcagaatttttcgattttagttgaaaataatgaggccaacgttccagcgctggcattctatatgagaagtgatcaaatagacgaatatctcggaaaggattcatcaaagataaaggagaattcgcctatagtggcgttgccgctattgtctgaatcgcaaggtattagaatgccctcaaacccggcattaccgaaaaaactattgaatgttccgtatgagctattagcaccgacaggtgactattttggattcaatatcataatcggtgggtttcatccaaattaccaatcctctaactttcattgttttatatacgatattaactccggaaagtggtcacgagtcgctaccgcctgccctgactgcgatatcaataaacatcgattctggagggtatttgtttggaaatcgcatcatcagacgattttgttgggtaccaagactgatgattattattcacccagtgtacaaagattcgaccatctttccacttttggattaccgctggtaaatatttttaacaagacgatccaattaccccatcacaagatctcagcttcgtctttaccaatacccatagaaaattttgcgaagcataaggatactccattaaaaaaggtgagcttcacttcatcagctacctcccaatttgaaaactacattagatatatagctccgcccttggaaatgtcttctatccaatctgtgtttccaccgtatgccatggttctaggtaaagacgctttagagatttacgggaagcctctttctgattttgaatttattactagtgaaggtgactccattggtataccatgctatttgctgaggaaaagatggggccgttatttcgacatgcttttatcccagagttatacaaaagtttgtgctgattatgaaactactgatacgcaatctacactaataaaattttcaccacattcatctagaaacagttctaaagctgtgaggcaagaaggtagactctcttcatcgggttcactggacaattatttcgaaaaaaacttcccaatctttgcgagaacaagtgtttccgaggcccagaacacacaacctcaggtagcgaatgctgatgcgaaagctccaaatacacctagtacgtcagatgaaccaagctcttcctcctcttccgatttatactccactcctcattaccaacgtaataatgatgaagaggatgacgaggacccggtttccccgaagcctgtatcaaaatcaaacagtatctatagacccattagaaagacagaaagttcatcaacaacgagttcttcaaacggtatgatttccagagtacctttcaaggaaaaagcggctgtaacgtcaaacactgaagccctcttggagtcaaacctttcacttcaagaattgagccgaagaagatcatcacttatgagcatcccgtctggtgaacttctaaggtcctctatatctgaagcggaacatcaacgtcgtgcatctcatccactcacttcatcaccgctctttgaagatagtggaaccccttgcggcaaacaactgcaacaactgcaacaacatactatacaaaatcctcataaccatttgtcgccccggaggttttcaagaagtgcaagaagttcaatttcctacgtgagttcttcttccgatagacgtggaaattcaatttctagcaggagcacaagcgatagctttggaactccaccagttttgggcgttttgaatgtaccattacctcctcaaacaagagaacctaatgaaccacctccaccatgtcctgcaatgagtaccggctcaaatacaaggcgaagcaacaccttaacggattatatgcattctaacaaggctagcccgttttcttctcgtagatcgtcgcatatcggtagacgttctagtacaccggaaacggaaaacgcattctctgcaacaccaagagcatccttagatggtcaaatgttaggaaaatctttgaaggaaggttctacatcacagtacacacagccaaggatgaactctttcccaaaggctaatgaaaccatacaaacacccacatcatcaaacaatgaatggagccgtcaatccgttacttcaaatacagatagttttgacagtttgcaatctaattttgcgttagagttggaaccacttttaacaccgaggtcactttatatgccttggccaacctccacagttcgagcatttgcggaatttttttacactgggcaagtaaacagtaaatggcttttggcaccagttgctcttgatttattagtaatggccaagatttatgaaataccattactgtataaattgatccttgaagttttatattcgattctggctaaaaaagaagaaagtttatccttaatatgtacttcgttaatggagacctttcgcaccaaaactctgaactcctataaaggtgatgaagaaaaaacaaatacttatttgacttcaaacgataactatcaggaactgttgaaattaaaagtgtcgctggagaatatcgacaatgggtattatgacccagatttgttacgcaaacagtcaagagcacagtcttcaagcacacaagaaagcagtggtagtgcaaacggcgaaaaaactgcaactggcgctggctctctggagacttcttcaaccaatgtccctacagtatttgctggcggtcccagagatagccacaattcagtaggctccattggttttccaaacagtatgaatatacaaggctcaagaagatcgacatcaggattttctccacgcgttaagatgaaatcgagcttaagcaaggaaatagatcccaaaactttttatgaagagtacgaaccaaaagagggcaaaagttttgatgataacgatgatcaacaaaccaacatcggaagttttaaccttcatttgtttgatatgaattatggttccatcagcagcagtagcactaacagcattagtagcagtgatttagaagagaaagaagaacaagagcaacttcaagatttattggaaatagaaagagaagattctgctgagatcttagacgcaagattcagaaacaaagaagatgataaagtgacgaaggatatatcaaatgacaagaaacgcaattacttaccgcatgaaaaaaataacttgaaagccaaggaaggtaaggaaactagagacgtaagggaggaggaggaagaattcgattttggtttgggaatgctttcgctcaataaaataaaaagagaagccaagcatgtcgataaggtggacgattctgtagaccctttatttaaatcatctgctttccctcaaagtcccattcgagcatacgggtctaccacaagaactagttcggcttctggaaagccattcagggacaatcgttcatttaatgcattttctgttttgacattagaaaatatggcgtctgcaaatgccctacctcccgttgattatgtcataaaatcaatatacagaaccactgtgttagtgaacgatatcagactaatggttcgttgcatggattgcattgaattatctaaaaatttacgagcgctcaagaaaaaaactatggaagatatttcgaaattgaaaggcatctctaaaccgtcaccatag

SEQ ID NO: 5 - DNA, 20 bp, synthetic construct
  source 1..20: mol_type = other DNA
gggtagcagaagcaagcgga

SEQ ID NO: 6 - DNA, 20 bp, synthetic construct
  source 1..20: mol_type = other DNA
gatgtatagcagcatattct

SEQ ID NO: 7 - DNA, 20 bp, synthetic construct
  source 1..20: mol_type = other DNA
ggatgagaatctgacaaagg

SEQ ID NO: 8 - DNA, 69 bp, synthetic construct
  source 1..69: mol_type = other DNA
taaggcagactccgtggagtgtaagagaagttcaaagcaaggttaggcttgtggtcggctggagatcgg

SEQ ID NO: 9 - DNA, 69 bp, synthetic construct
  source 1..69: mol_type = other DNA
gaatttgaattgtcctgagctgacctggtctgcccgcttcgataaactgcagccgttatggcgggcatc

SEQ ID NO: 10 - DNA, 22 bp, synthetic construct
  source 1..22: mol_type = other DNA
aaagtctatttcaagttcacag

SEQ ID NO: 11 - DNA, 30 bp, synthetic construct
  source 1..30: mol_type = other DNA
tctagacatcaagtctaagaaaaacgtctc

SEQ ID NO: 12 - DNA, 30 bp, synthetic construct
  source 1..30: mol_type = other DNA
tctagacatcaagtctaagaaaaacatctc

SEQ ID NO: 13 - DNA, 25 bp, synthetic construct
  source 1..25: mol_type = other DNA
gtttttcttagacttgatgtctaga

SEQ ID NO: 14 - DNA, 29 bp, synthetic construct
  source 1..29: mol_type = other DNA
cgctttttccttgaaaggtactctgaaaa

SEQ ID NO: 15 - DNA, 29 bp, synthetic construct
  source 1..29: mol_type = other DNA
cgctttttccttgaaaggtactctggaaa

SEQ ID NO: 16 - DNA, 25 bp, synthetic construct
  source 1..25: mol_type = other DNA
cagagtacctttcaaggaaaaagcg

SEQ ID NO: 17 - DNA, 30 bp, synthetic construct
  source 1..30: mol_type = other DNA
aaggtctccatcaacgaagtacatattaag

SEQ ID NO: 18 - DNA, 30 bp, synthetic construct
  source 1..30: mol_type = other DNA
aaggtctccatcaacgaagtacatactaag

SEQ ID NO: 19 - DNA, 25 bp, synthetic construct
  source 1..25: mol_type = other DNA
tatgtacttcgttgatggagacctt

SEQ ID NO: 20 - DNA, 18 bp, synthetic construct
  source 1..18: mol_type = other DNA
ggacgtagcggtctatgg

(19) United States
(12) Patent Application Publication
(10) Pub. No.: US 2025/0051699 A1
(43) Pub. Date: Feb. 13, 2025

(54) METHODS OF PREVENTING INHIBITION OF FLAVOUR PRODUCTION IN YEAST

(71) Applicant: Anheuser-Busch InBev S.A., Brussels (BE)

(72) Inventors: Philippe MALCORPS, Marchin (BE); Stijn DE GRAEVE, Bertem (BE); Ben SOUFFRIAU, Heverlee (BE); Arne HAGMAN, Malmo (SE); Johan THEVELEIN, Blanden (BE)

(21) Appl. No.: 18/721,140

(22) PCT Filed: Dec. 16, 2022
(86) PCT No.: PCT/EP2022/086516

§ 371 Date: Jun. 17, 2024

(30) Foreign Application Priority Data
Dec. 17, 2021 (BE) BE2021/5999

(51) Int. Cl. C12C 12/00; C07K 14/395

(52) U.S. Cl. CPC C12C 12/004; C07K 14/395; C12C 12/006; C12C 2200/05

(57) ABSTRACT

The present invention relates to the field of fermentation and in particular to methods of improving the flavour and quality of beer produced by fermentation.

### FIELD OF THE INVENTION

The present invention relates to the field of fermentation and in particular to methods of improving the flavour and quality of beer produced by fermentation.

### BACKGROUND OF THE INVENTION

Beer is the most consumed alcoholic beverage worldwide. It is traditionally made from four key ingredients: malted cereals (barley or other), water, hops, and yeast. Each of these ingredients contributes to the final taste and aroma of beer. During fermentation, yeast cells convert cereal-derived sugars into ethanol and CO₂. At the same time, hundreds of secondary metabolites that influence the aroma and taste of beer are produced. Variation in these metabolites across different yeast strains is what allows yeast to so uniquely influence beer flavour (Maicas, Microorganisms, 2020).

Recently, as a result of industrialization and company growth, beer breweries have been able to brew larger and larger quantities of beer. This has prompted a shift from horizontal open vessels to deep, vertical cylindroconical tanks used for yeast fermentation at large commercial scale. Unexpectedly, this new fermentor design resulted in compromised yeast growth, sluggish fermentation, poor diacetyl stripping and insufficient fruity flavours in the beer. The latter is caused by inadequate ester production by the yeast, mainly of isoamyl acetate, a key aroma compound responsible for the fruity “banana” flavour of beer. The main inhibiting agent of isoamyl acetate productivity turned out to be the high level of dissolved CO₂ in the cylindroconical tanks, which increases proportionally with the hydrostatic pressure at increasing depths of the fermentor. Large-scale beer production is performed in cylindroconical tanks with depths reaching 10 to 18 meters, leading to hydrostatic pressures of approximately 1.0-1.8 bar (approximately 1.0-1.8 atmospheric pressure units). When yeast is subjected to CO₂ pressure during alcoholic fermentation the formation of fusel alcohols and acetate esters is strongly inhibited. At CO₂ pressures above 0.5 bar, the exponential growth rate starts to be reduced, with complete inhibition at 2.7 bar. A CO₂ overpressure of 1 bar corresponds approximately to the hydrostatic pressure at 10 meters depth, and causes a drop in isoamyl acetate production from 3.6 to 1.4 mg/L, which compromises beer quality.

The cause of the CO₂ inhibition of yeast flavour production has always remained enigmatic. There therefore exists a need to identify the cause of flavour inhibition and improve beer quality. The present invention addresses this need.

### SUMMARY OF THE INVENTION

We have identified a unique allele that confers tolerance to inhibition of yeast flavour production by high carbon dioxide pressure. We also show through CRISPR/Cas mediated allele exchange these superior alleles can be used successfully to enhance tolerance of yeast flavour production to high carbon dioxide pressure in commercial lager brewing strains, creating cisgenic superior industrial strains that are not significantly affected in other traits important for brewing.

In one aspect of the invention, there is provided a method of increasing the flavour of a fermented product, wherein the method comprises using a genetically altered yeast in fermentation and obtaining the fermented product, wherein the yeast is characterised by at least one mutation in at least one MDS3 gene.

In another aspect there is provided a method of producing a fermented product, wherein the method comprises using a genetically altered yeast in fermentation and obtaining the fermented product, wherein the yeast is characterised by at least one mutation in at least one MDS3 gene.

In another aspect there is provided a fermented product obtained or obtainable by the methods of the invention.

In another aspect there is provided a genetically altered yeast, wherein the yeast is characterised by at least one mutation in at least one MDS3 gene.

In another aspect there is provided a genetically altered yeast, wherein the yeast comprises a nucleic acid construct comprising a nucleic acid sequence encoding a MDS3 polypeptide as defined in SEQ ID NO: 3 or a fragment or variant thereof.

In another aspect there is provided the use of the genetically altered yeast of the invention in fermentation.

In another aspect there is provided the use of the genetically altered yeast of the invention to increase the flavour of a fermented product.

In another aspect there is provided an isolated nucleic acid encoding the amino acid sequence of SEQ ID NO: 3 or a variant thereof.

In another aspect there is provided a nucleic acid construct comprising a nucleic acid sequence encoding a MDS3 polypeptide as defined in SEQ ID NO: 4 or a fragment or variant thereof, wherein the nucleic acid sequence is operably linked to a regulatory sequence.

In another aspect there is provided a yeast comprising the nucleic acid construct of the invention.

In another aspect there is provided a method for identifying and selecting a yeast that is capable of increasing the flavour of a fermented product, the method comprising detecting in a yeast genome at least one polymorphism in at least one MDS3 gene and selecting said yeast.

In another aspect there is provided a method of producing a genetically altered yeast of the invention, the method comprising introducing at least one mutation into at least one MDS3 gene using genome editing.

In another aspect there is provided a method for producing a hybrid yeast capable of increasing the flavour of a fermented product, the method comprising introducing at least one mutation into at least one MDS3 gene of a first haploid yeast and hybridising the first haploid yeast cell or spore with a second haploid yeast cell or spore to produce a hybrid yeast.

In another aspect there is provided a method for producing a hybrid yeast capable of increasing the flavour of a fermented product, the method comprising identifying and selecting a yeast that is capable of increasing the flavour of a fermented product, the method comprising detecting in a yeast genome screen at least one polymorphism in at least one MDS3 gene, selecting said first yeast and hybridising a haploid of the first yeast cell or spore with a second haploid yeast cell or spore to produce a hybrid yeast.

In another aspect there is provided a hybrid yeast obtained or obtainable by the methods of the invention.

### DESCRIPTION OF THE FIGURES

The invention is now described in the following non-limiting figures:

FIG. 1 shows the lab-scale set-up for high CO₂ pressure fermentation. A. Scheme of the lab-scale high CO₂ pressure fermentation system. B, C IAAc/Alc ratio after fermentation with four brewing yeast strains with and without extra CO₂ pressure: B. +0.50bar, C. +0.65bar, and D. Fermentation progress (apparent extract, in ° Plato) with and without extra CO₂ pressure (+0.65bar). Strain JT28332 is an ale yeast and strains JT28333-JT28335 are Frohberg lager yeasts.

FIG. 2 shows screening for yeast strains producing superior IAAc/Alc ratios in high CO₂ pressure fermentations. A. Frequency distribution of the IAAc/Alc ratio in 200 preselected Saccharomyces strains obtained with gas chromatography analysis of fermentations in malt extract media under 0.65bar extra CO₂ pressure. The broken gray line indicates the cut-off for the 16 strains with the highest IAAc/Alc ratio (>0.06). B. Residual maltose level after 4 days of fermentation in malt extract medium (15° P, non-aerated) for 120 segregants of strain JT22329 (Kyokai no. 1). The broken gray line indicates the 49 segregants with the lowest residual maltose level that were subsequently subjected to flavor profiling in pressurized beer fermentations. C. Scatter plot of isoamyl acetate and isoamyl alcohol levels produced in 0.65bar pressurized beer fermentations with maltose fermenting segregants of JT22329. Dotted lines represent the average IAAc/Alc ratios (0.04, 0.06 and 0.08). The symbol of the selected superior segregant is shown in green and symbols of unrelated inferior haploid strains are indicated in red. Arrows indicate the selected superior segregant (Seg.63), the unrelated inferior haploid strain (ER7A) selected for mating, and the diploid parental strain (JT22329) for fermentations with and without extra CO₂ pressure. D. Frequency distribution of IAAc/Alc ratios obtained from gas chromatography analysis of fermentations in malt extract medium with 0.65bar extra CO₂ pressure for 185 haploid segregants of the hybrid diploid Seg.63/ER7A preselected for efficient maltose fermentation. The ratios have been normalized per fermentation batch to that of strain JT22329. The haploid parental strains, Seg.63 and ER7A, were included in each of the eight fermentation batches, and are shown on top with 10-90 percentile box plots. The broken gray line indicates the 100% cut-off for the 76 segregants selected for confirmation. E. Confirmation and selection of the haploid segregants of Seg.63/ER7A showing the highest IAAc/Alc ratio. The ratios have been normalized per fermentation batch to that of strain JT22329. The dotted gray line indicates the 106% cut-off for the 28 segregants with the highest IAAc/Alc ratio, selected for the superior pool (shown in blue). The haploid parental strains, ER7A and Seg.63, included in each of the 6 batches, are shown in green and red, respectively.

FIG. 3 shows Identification of MDS3 as the major causative gene in QTL2. QTL2 was the most strongly linked QTL identified for superior IAAc/Alc ratio in fermentations under CO₂ pressure. To narrow down further the area in QTL2, we performed bulk RHA analysis with three blocks of genes. A. Bulk RHA results for QTL2, identifying block 1 as causative. The position of the blocks is indicated on top. B. To identify the causative genetic element in block 1 of QTL2, individual gene RHA was performed. Missense (single asterisk), and all combined promoter (P) and terminator (T) mutations are indicated for the genes in block 1. The significance indicated above the bars was determined with a student t-test with correction for multiple testing with false discovery rate of 1%. * p<0.05, ** p<0.01, *** p<0.001. The fermentations were carried out in quadruplicate with duplicate fermentations for each of two independent isolates. 20

FIG. 4 shows allele replacement of the MDS3 gene in the parental strains. The causative effect of the MDS3 alleles isolated from the superior Seg.63 and inferior ER7A strain was confirmed by allele replacement using CRISPR/Cas9 technology. IAAc/Alc ratio in strains obtained by allele replacement in the Seg.63 and ER7A haploid parent strains (A.) and in the Seg.63/ER7A hybrid diploid strain (B.). Significance indicated above the bars was determined with a student t-test with correction for multiple testing with false discovery rate of 1%. * p<0.05, ** p<0.01, *** p<0.001. The fermentations were carried out in triplicate.

FIG. 5 shows engineering of the MDS3^{Seg 63} allele into the tetraploid lager yeast JT28325. Allele replacement was performed with one or with all four alleles in a Frohberg type lager yeast. Significance indicated above the bars was determined with a student t-test with correction for multiple testing with false discovery rate of 1%. * p<0.05 and ** p<0.01. The fermentations were carried out in triplicate with three independent transformants.

FIG. 6 shows identification of F724S (T2171C) as the causative SNP variant in MDS3^{Seg 63}. To identify the causative SNP(s) in the MDS3 gene, we reintroduced mutant alleles assembled from DNA fragments of MDS3 containing all possible combinations of SNPs. Significance of the difference with the ER7A reference strain indicated above the bars was determined with a One-way ANOVA test with Dunett's correction for multiple testing. * p<0.05, ** p<0.01, *** p<0.001. The fermentations were carried out in triplicate.

FIG. 7 shows statistical comparison of flavour compound levels produced in CO₂ pressurized fermentations by the wild-type ER7A MDS3WT strain and strains containing missense mutations in the MDS3 gene. Fermentations were carried out in triplicate with malt extract media and 0.65 bar CO₂ overpressure. Metabolites were measured with GC-FID. P-values from unpaired t-tests with Holm-Sidak correction for multiple testing, were obtained by comparing the values of the reference strain ER7A with those of a strain with either re-insertion of the wild-type MDS3 allele or an MDS3 allele with a combination of the missense SNPs C305T (T102M), T2171C (F724S), and A3229G (11077V), or the complete MDS3 allele from Seg.63. The Seg.63 superior haploid strain was included as a positive control. Compounds are indicated with abbreviations as follows: AAld, Acetaldehyde; IbAlc, Isobutanol; laAlc, Isoamyl alcohol; PhAlc, Phenyl ethanol; EAc, Ethyl acetate; IbAc, Isobutyl acetate; IAc, Isoamyl acetate; PhAc, Phenylethyl acetate; EtHex, Ethyl hexanoate; EtOct, Ethyl octanoate; EtDec, Ethyl decanoate.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of microbiology, molecular biology, chemistry, biochemistry, recombinant DNA technology, and bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The CO₂ inhibition of flavour production in large cylindroconical fermentors has long been recognized as one of the most important issues at industrial scale in modern brewing. Nevertheless, yeast strains maintaining appropriate flavour production under high CO₂ pressure have not yet been developed for industrial brewing conditions.

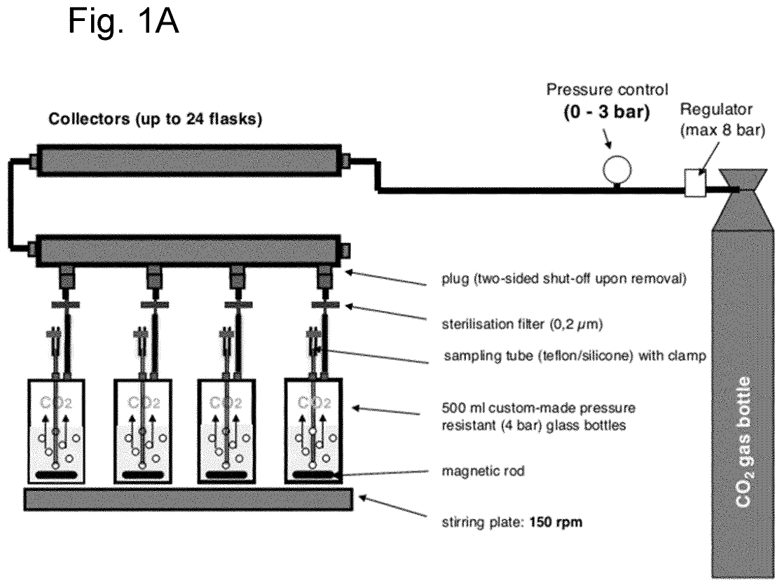

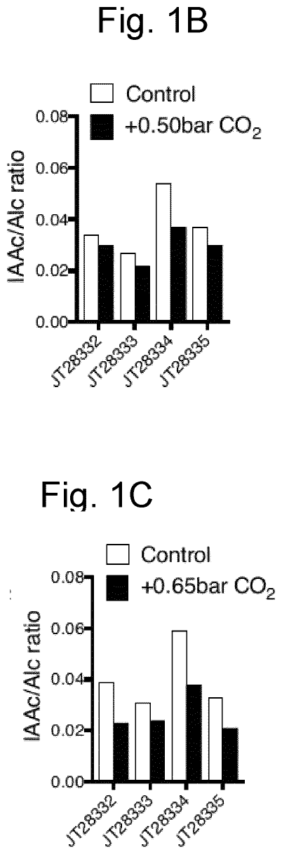

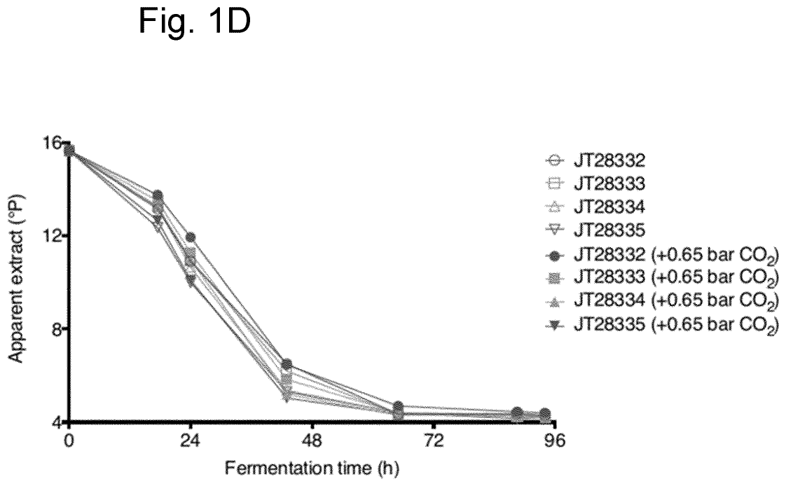

We have first developed a laboratory scale system to perform fermentations under high CO₂ pressure, mimicking the large-scale industrial fermentation conditions (FIG. 1). Using our laboratory scale fermentation system, we observed a reproducible, strong and strain-dependent inhibition of the AATase activity (as reflected in the IAAc/Alc ratio) at 0.65 bar CO₂ pressure, with minimal inhibition on fermentation performance, and fermentation under higher CO₂ pressures was therefore not investigated further in this study.

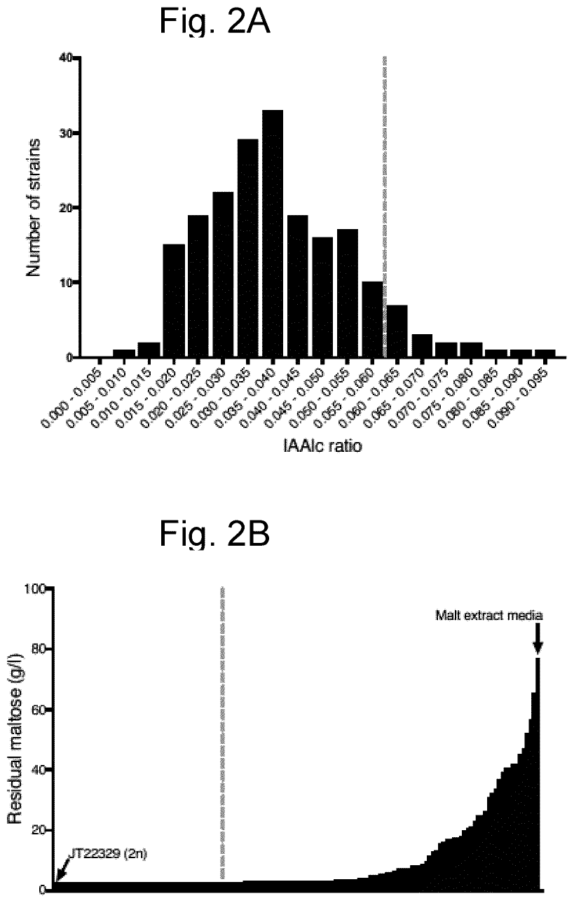

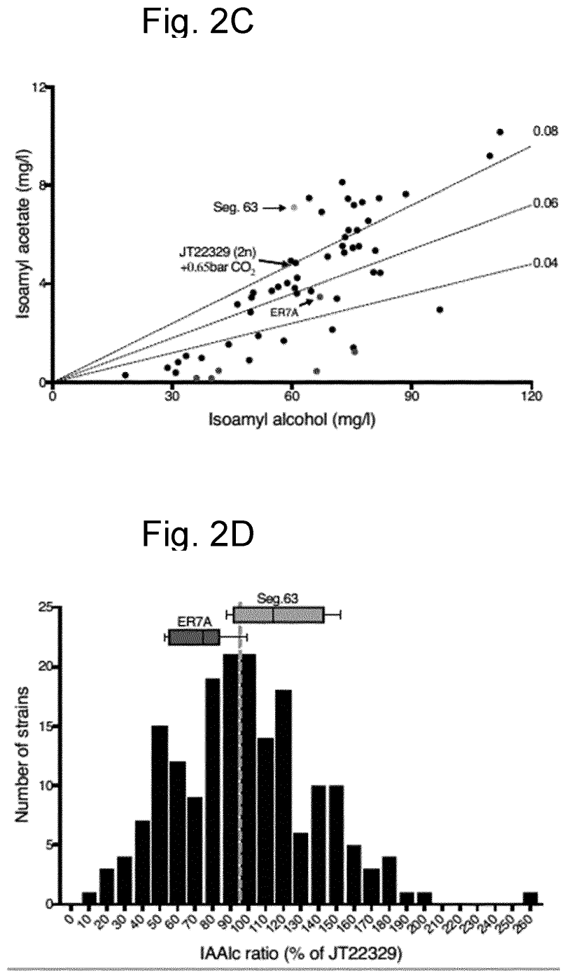

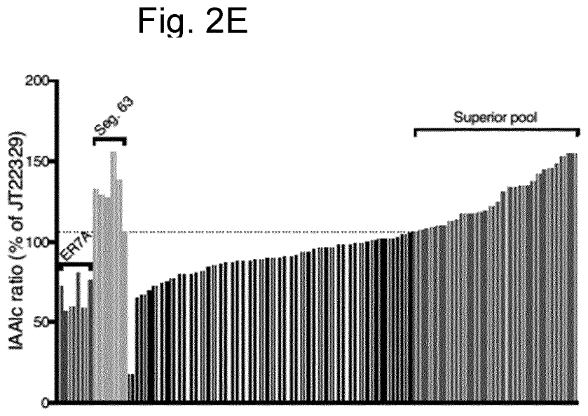

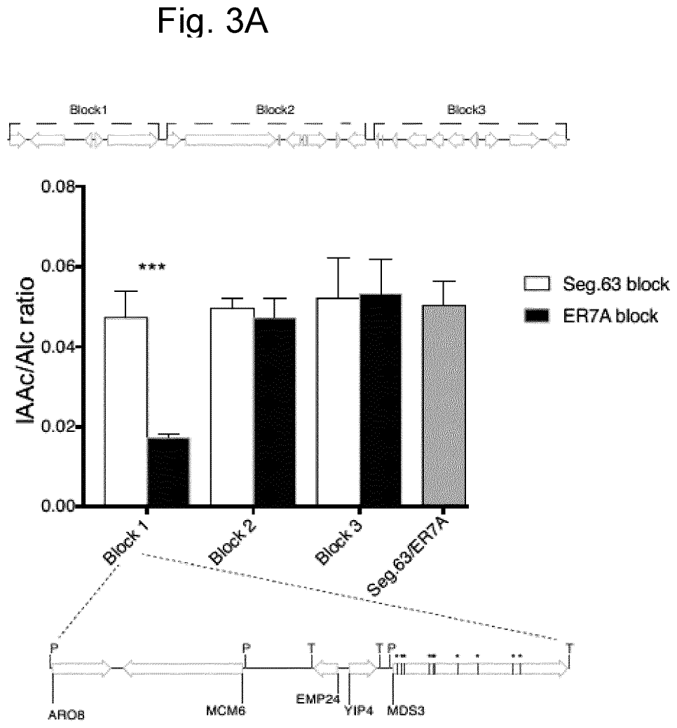

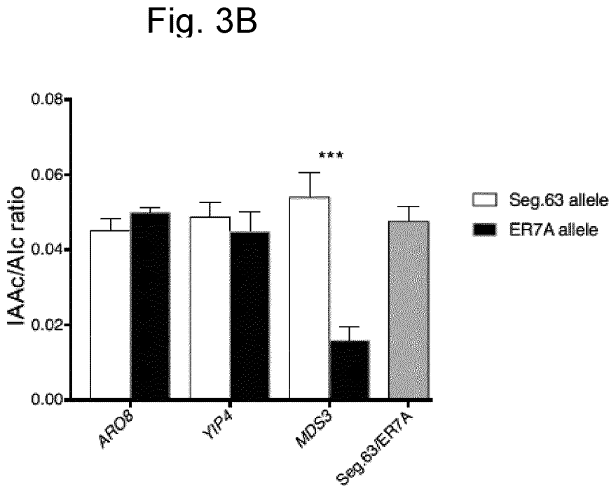

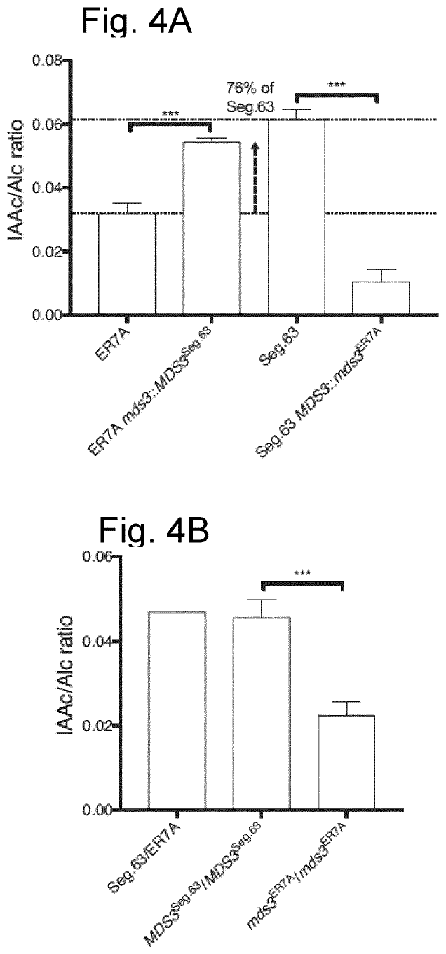

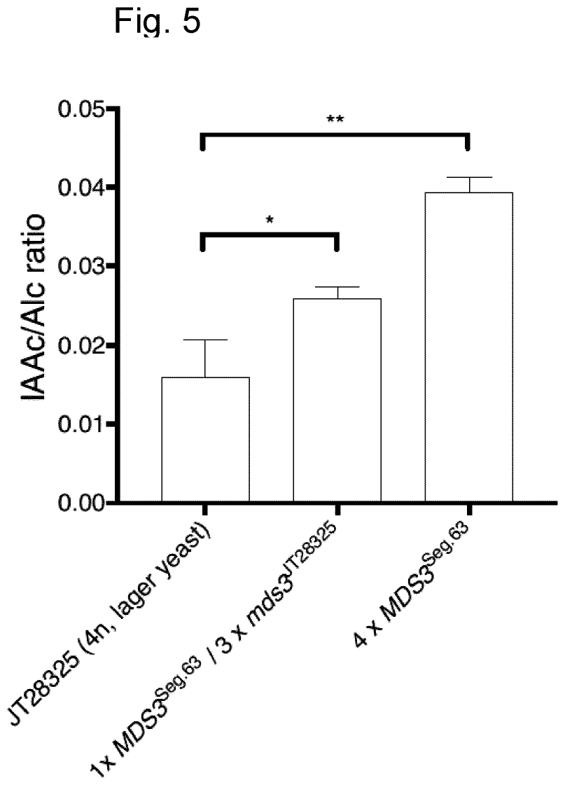

Using this set-up, we then screened our S. cerevisiae strain collection for strains displaying high tolerance to CO₂-induced reduction of the IAAc/Alc ratio and observed a strong variation, with strains performing clearly better than commercial brewing strains (FIG. 2). Using the most superior strain, we next applied the polygenic analysis platform, consisting of QTL mapping by pooled-segregant whole genome sequence analysis and SNP coverage mapping, followed by reciprocal hemizygosity analysis. This led to the identification of a novel natural allele of the MDS3 gene (FIG. 3), in which we pinpointed a single SNP variant, T2171C, as being the sole SNP in MDS3 responsible for the increase in the IAAc/Alc ratio by the MDS3^{Seg 63} allele (FIG. 4). Engineering of the novel MDS3 allele into a tetraploid lager yeast by allele exchange enhanced the IAAc/Alc ratio (isoamyl acetate production yield) by 145% in a beer fermentation under elevated CO₂ pressure (FIG. 5).

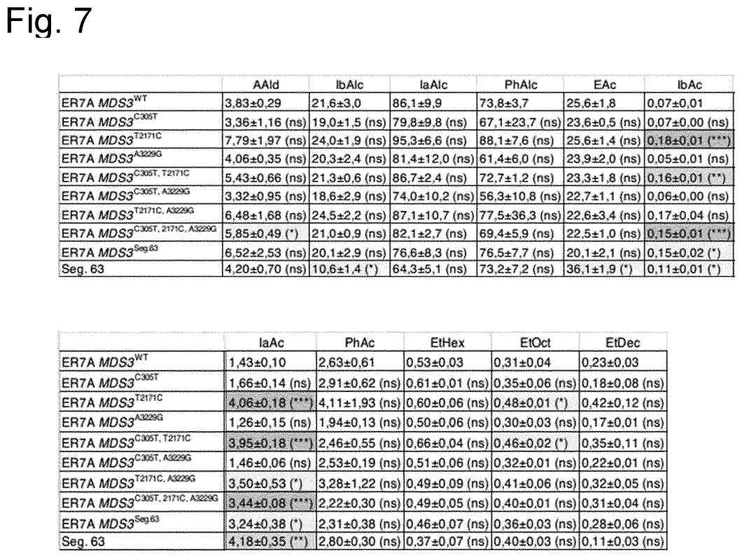

We have demonstrated that the superior variant of MDS3, MDS3^{T2171C}, specifically increases the isoamyl and isobutyl acetate levels and does not affect the production of other aroma compounds, as shown in FIG. 7. These acetate esters are uniquely formed through esterification of the fusel alcohol precursors isoamyl and isobutyl alcohol by the AATase enzymes Atf1 and Atf2 using acetyl-coA as a co-substrate. We suggest that the MDS3^{T2171C} (Mds3^{F724S}) variant might act by elevating the expression level of the AATase genes. The superior haploid Seg.63 segregant and the hybrid Seg.63/ER7A diploid showed a growth and fermentation defect when they were downgraded with the MDS3ER7A allele. This indicates that the MDS3 gene plays a vital role in at least one important cellular pathway, different from those involved in regulation of isoamyl acetate production, and that this effect is dependent on the genetic background of the strain.

Accordingly, in one aspect of the invention, there is provided a method of increasing the flavour of a fermented product, wherein the method comprises using a genetically altered yeast in fermentation, wherein the yeast is characterised by at least one mutation in at least one MDS3 gene. Preferably, the method comprises obtaining the fermented product.

In another aspect of the invention, there is provided a method of producing a fermented product, wherein the method comprises using a genetically altered yeast in fermentation and obtaining the fermented product, wherein the yeast is characterised by at least one mutation in at least one MDS3 gene.

In one embodiment, the flavour is a banana flavour. More preferably, the flavour of a fermented product is increased by increasing isoamyl acetate and/or isobutyl acetate levels. Isoamyl and/or isobutyl acetate is uniquely produced by the yeast alcohol acetyl coenzyme A (acetyl-coA) transferase enzymes (AATases), Atf1 and Atf2, by condensation of the precursor molecules isoamyl alcohol and acetyl-coA. Atf1 is responsible for the majority of AATase activity for production of the flavor-active acetate esters, with isoamyl acetate levels being more than 80% reduced by ATF1 deletion and increased 180-fold by constitutive overexpression of ATF1 in a laboratory yeast. The major limiting factor for production of isoamyl acetate is the expression level of ATF1, which correlates with the final concentration of isoamyl acetate in beer. Acetate ester production by Atf1 is regulated by a number of factors. It is inhibited by dissolved oxygen and low nitrogen content, and enhanced by high gravity (i.e. high sugar level), whereas the effects of fermentation temperature and pitching rate appear to be strain dependent. Accordingly, in one example, an increase in isoamyl acetate production can be measured as an increase in the ratio of isoamyl acetate (IAAc) to isoamyl alcohol (Alc).

Fujiwara et al. have performed molecular characterization of the ATF1 promoter elements and showed that activation by the Rap1 repressor/activator transcription factor is essential for ATF1 expression. Rap1 has roles in many cellular responses, including induction of ribosomal genes during growth. Recently, a truncated allele of the Tor1 kinase was shown to enhance the levels of acetate esters in alcoholic fermentations. TOR (Target Of Rapamycin) consists of the Tor1 and Tor2 kinases, which form the TORC1 and TORC2 complexes, that regulate various cellular functions. TORC1 contains either Tor1 or Tor2, and TORC2 exclusively Tor2. Disruption of Tor1 only affects the growth of yeast, whereas Tor2 is essential. TORC1 functions in a major growth promoting signal transduction pathway and acts as regulator of nitrogen catabolite repression genes. Fujiwara et al. found that ATF1 expression is hampered by deletion of the downstream TORC1 effector kinase Sch9, linking the TORC1 pathway directly to ATF1 expression. On the other hand, it has also been shown that high activity of the protein kinase A (PKA) signalling pathway, another major growth activator, increases the transcript level of the ATF1 gene.

As used herein a “fermented product” may refer to the product produced by fermentation of, for example, crops and products thereof including grain or fruits. In one example, the fermented product is a fermented beverage such as beer.

In one embodiment isoamyl and/or isobutyl acetate production is increased by at least 50%, 60%, 70%, 80%, 90%, 100%, 110%, 120%, 130%, 140%, 150%, 160%, 170%, 180%, 190% or at least 200% when the genetically altered yeast of the invention was used in fermentation, compared to when a non-genetically altered yeast is used (e.g. a yeast that does not contain a mutation in the MDS3 gene). More preferably, isoamyl and/or isobutyl acetate production is increased between 100 and 150%, and even more around 150%.

In one embodiment, the method comprises culturing the yeast under high carbon dioxide conditions. As use herein, “high carbon dioxide” conditions may be considered at least 0.5 bar, preferably at least 0.65 bar. In another embodiment, “high carbon dioxide” conditions may be considered between 1 and 1.8 bar.

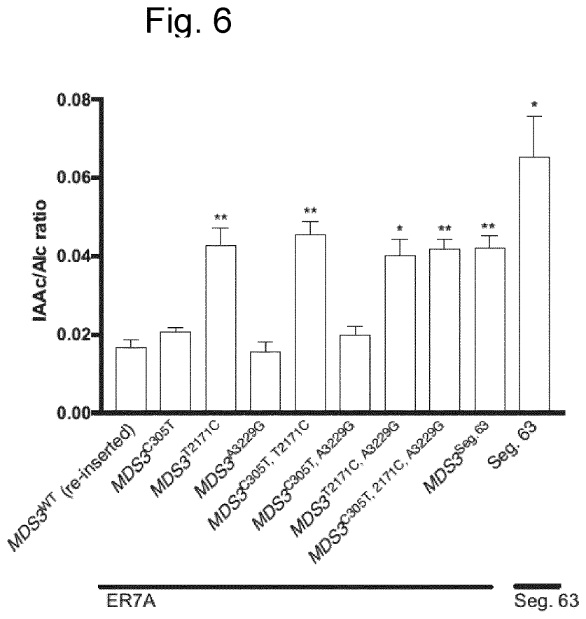

By “at least one mutation in at least one gene” is meant that where the gene is present as more than one copy or homeoallele/homeologue (with the same or a slightly different sequence) there is at least one mutation in at least one gene. In one embodiment, all copies of the gene are mutated. As shown in FIG. 6, mutation of one allele in tetraploid yeast resulted in a 61% rise in the IAAc/Alc ratio. Mutation of all four alleles in tetraploid yeast resulted in a 145% rise in the IAAc/Alc ratio.

Mds3 is also known as a negative regulator of sporulation MDS3. Mds3 is understood to be a positive regulator of the Target Of Rapamycin (TOR) pathway, since a mds3 knock-out S. cerevisiae strain is highly sensitive to rapamycin, a TORC1 inhibitor. In Candida albicans, Mds3 interacts with the downstream TORC1 effector and PP2A-related protein phosphatase Sit4, linking the Mds3 protein to nutrient sensing through TORC1. High TOR activity is correlated with strong growth, partly due to high expression of ribosomal genes involved in translation. The major AATase ATF1 gene is strongly regulated by the Rap1 activator/repressor transcription factor, and disruption of the downstream TORC1 effector, Sch9, reduces its expression level.

In one embodiment, the MDS3 gene comprises a nucleic acid sequence that encodes a MDS3 polypeptide as defined in SEQ ID NO: 3 or a homologue or functional variant thereof. In a further embodiment, the MDS3 gene comprises or consists of a nucleic acid sequence as defined in SEQ ID NO: 2 or a homologue or functional variant thereof.

The term “functional variant” (or “variant”) as used herein with reference to any of SEQ ID Nos refers to a variant sequence or part of the sequence which retains the biological function of the full non-variant sequence. A functional variant also comprises a variant of the MDS3 which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes that result in substitution of one negatively charged residue for another, such as aspartic acid for glutamic acid, or one positively charged residue for another, such as lysine for arginine, can also be expected to produce a functionally equivalent product. Nucleotide changes that result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homologue, as used herein, also designates a MDS3 gene orthologue from other yeast species. A homologue may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by any of the SEQ ID Nos identified herein. Functional variants of MDS3 gene homologues as defined above are also within the scope of the invention.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologues can be identified by sequence comparisons and identifications of conserved domains. There are predictors in the art that can be used to identify such sequences. The function of the homologue can be identified as described herein and a skilled person would thus be able to confirm the function, for example, when expressed in yeast.

Thus, the nucleotide sequences of the invention and described herein can also be used to isolate corresponding sequences from other organisms, particularly other yeast. In this manner, methods such as PCR, hybridization, and the like can be used to identify such sequences based on their sequence homology to the sequences described herein. Topology of the sequences and the characteristic domain structure can also be considered when identifying and isolating homologues. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen yeast. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2nd ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

In a further embodiment, a functional variant as used herein can comprise a nucleic acid sequence encoding a MDS3 polypeptide as defined herein that is capable of hybridising under stringent conditions as defined herein to a nucleic acid sequence as defined in SEQ ID NO: 2.

In one embodiment, there is provided a method of increasing the flavour of a fermented product, wherein the method comprises using a genetically altered yeast in fermentation, wherein the yeast is characterised by at least one mutation in at least one MDS3 gene, wherein the MDS3 gene comprises or consists of
a. a nucleic acid sequence encoding a polypeptide as defined in SEQ ID NO: 1; or
b. a nucleic acid sequence as defined in SEQ ID NO: 2; or
c. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
d. a nucleic acid sequence encoding a MDS3 polypeptide as defined herein that is capable of hybridising under stringent conditions as defined herein to the nucleic acid sequence of any of (a) to (c).

In one embodiment, the at least mutation is selected from an insertion, a deletion or an substitution. As used herein, an “insertion”, “deletion” or “substitution” may refer to the insertion, deletion or substitution of at least one, two, three, four, five, six, seven, eight, nine or ten nucleotides. The mutation may be in a coding or non-coding portion of the gene. Alternatively, the mutation may be in the MDS3 promoter, wherein the mutation affects the expression of the MDS3 gene. Preferably, the mutation is a dominant mutation. As such, the skilled person would understand that any mutation that leads to a dominant phenotype (i.e. increased levels of isoamyl and isobutyl acetate) would fall within the scope of the invention.

In one specific embodiment, said mutation may comprise the substitution of at least one of the following:
T to M at position 102 of SEQ ID NO: 1 or a homologous position in a homologous sequence;
F to S at position 724 of SEQ ID NO: 1 or a homologous position in a homologous sequence; and/or
I to V at position 1077 of SEQ ID NO: 1 or a homologous position in a homologous sequence.

In a preferred embodiment, the mu tation is at least a single substitution of F to S at position 724 of SEQ ID NO: 1 or a homologous position in a homologous sequence. This correlates to a T to C mutation at position 2171 of SEQ ID NO: 2 or a homologous position in a homologous sequence.

In an alternative embodiment, the mutation is the introduction of one or more MDS3^{Seg63} alleles, wherein preferably the MDS3^{Seg63} allele encodes a MDS3 polypeptide as defined in SEQ ID NO: 3 or a functional variant or homologue thereof. More preferably, the MDS3^{Seg63} allele comprises or consists of a nucleic acid sequence as defined in SEQ ID NO: 4 or a functional variant or homologue thereof. The MDS3^{Seg63} allele may be introduced such that it is operably linked to a suitable regulatory sequence, such as the endogenous MDS3 promoter. Alternatively, the MDS3^{Seg63} allele is swapped for a wild-type allele. Suitable methods for allele swapping are well known in the art, and include hybridisation and genome editing techniques such as CRISPR.

The C. albicans MDS3 gene has been linked to nutrient and pH dependent pseudohyphae formation and has been reported as being essential for growth in alkaline minimal medium (together with its paralogue PMD1), suggesting a role of MDS3 in pH regulation (39, 42). The growth inhibitory effects of CO₂ are to some extent caused by direct inhibition of metabolic enzymes, lowering of the intracellular pH and impairing mitochondrial function once the CO₂ (and bicarbonate) is inside the cells (4, 16-18). It is tempting to speculate that the Mds3^{F724S} allele provides tolerance to lower intracellular pH and sustains high TOR activity in the presence of high levels of dissolved CO₂.

In one embodiment, the mutation is introduced using mutagenesis or targeted genome modification.

In a preferred embodiment, the mutation is introduced using genome editing, preferably CRISPR. The use of this technology in genome editing is well described in the art, for example in U.S. Pat. No. 8,697,359 and references cited herein. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases, is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. Accordingly, using techniques known in the art it is possible to design sgRNA molecules that targets a MDS3 gene or promoter sequence as described herein. Tools for designing gRNA in yeast include CRISPy, CRISPy-web, CRISPR-ERA, Yeastriction and CHOPCHOP v2.

Alternatively, Cpf1, which is another Cas protein, can be used as the endonuclease. Cpf1 differs from Cas9 in several ways: Cpf1 requires a T-rich PAM sequence (TTTV) for target recognition, Cpf1 does not require a tracrRNA, and as such only crRNA is required unlike Cas9 and the Cpf1-cleavage site is located distal and downstream to the PAM sequence in the protospacer sequence. Furthermore, after identification of the PAM motif, Cpf1 introduces a sticky-end-like DNA double-stranded break with several nucleotides of overhang. As such, the CRISPR/Cpf1 system consists of a Cpf1 enzyme and a crRNA.

Cas9 and Cpf1 expression plasmids for use in the methods of the invention can be constructed as described in the art. Cas9 or Cpf1 and the one or more sgRNA molecule may be delivered as separate or as a single construct. Where separate constructs are used for the delivery of the CRISPR enzyme (i.e. Cas9 or Cpf1) and the sgRNA molecule(s), the promoters used to drive expression of the CRISPR enzyme/sgRNA molecule may be the same or different. In one embodiment, RNA polymerase (Pol) II-dependent promoters can be used to drive expression of the CRISPR enzyme. In another embodiment, Pol III-dependent promoters, such as U6 or U3, can be used to drive expression of the sgRNA.

In one embodiment, the method uses a sgRNA to introduce a targeted SNP or substitution mutation, in particular one of the substitutions described herein, into the MDS3 gene. As explained below, the introduction of a template DNA strand, following a sgRNA-mediated snip in the double-stranded DNA, can be used to produce a specific targeted substitution in the gene using homology directed repair. In another example, sgRNA (for example, as described herein) can be used with a modified Cas9 protein, such as nickase Cas9 or nCas9 or a “dead” Cas9 (dCas9) fused to a “Base Editor”-such as an enzyme, for example a deaminase such as cytidine deaminase, or TadA (IRNA adenosine deaminase) or ADAR or APOBEC. These enzymes are able to substitute one base for another. As a result no DNA is deleted, but a single substitution is made.

The genome editing constructs may be introduced into a yeast cell using any suitable method known to the skilled person. In an alternative embodiment, any of the nucleic acid constructs described herein may be first transcribed to form a preassembled Cas9-sgRNA ribonucleoprotein and then delivered to at least one yeast cell using any of the above described methods, such as lipofection, electroporation, biolistic bombardment or microinjection.

In another example, mutagenesis is physical mutagenesis, such as application of ultraviolet radiation, X-rays, gamma rays, fast or thermal neutrons or protons. In another example, by chemical mutagenesis is meant mutagenizing a yeast population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz (a) anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde.

In an alternative aspect of the invention, there is provided a method of increasing the flavour of a fermented product, wherein the method comprises using a genetically altered yeast in fermentation, wherein the yeast is characterised by the introduction or expression of a nucleic acid construct, wherein the construct comprises a nucleic acid sequence encoding a MDS3 polypeptide as defined in SEQ ID NO: 3 or a functional variant or homologue thereof as defined above. Preferably, the nucleic acid sequence is operably linked to a regulatory sequence, for example a promoter, such as a constitutive promoter.

The term “operably linked” as used herein refers to a functional linkage between the promoter sequence and MDS3 nucleic acid sequence, such that the promoter sequence is able to initiate transcription of MDS3.

In another alternative aspect of the invention, there is provided a method of producing a fermented product, wherein the method comprises using a genetically altered yeast in fermentation and obtaining the fermented product, wherein the yeast is characterised by the introduction or expression of a nucleic acid construct, wherein the construct comprises a nucleic acid sequence encoding a MDS3 polypeptide as defined in SEQ ID NO: 3 or a functional variant or homologue thereof as defined above.

In another aspect of the invention, there is provided a fermented product obtained or obtainable by the method of the invention.

In another aspect of the invention there is provided a genetically altered yeast, wherein the yeast is characterised by at least one mutation in at least one MDS3 gene as described above.

In another aspect of the invention, there is provided an isolated nucleic acid encoding the amino acid sequence of SEQ ID NO: 3 (the MDS3^{Seg63} allele) or a functional variant or homologue thereof. In a preferred embodiment, the isolated nucleic acid sequence comprises SEQ ID NO: 4 or a functional variant or homologue thereof.

In a further aspect of the invention, there is provided a nucleic acid construct encoding a MDS3 polypeptide as defined in SEQ ID NO: 3 or a fragment or variant thereof, wherein the nucleic acid sequence is operably linked to a regulatory sequence. Preferably the regulatory sequence is a constitutive promoter.

In another aspect of the invention, there is provided a genetically modified yeast cell transformed with the nucleic acid construct. The nucleic acid construct may be stably incorporated into the yeast genome. The terms “introduction”, “transfection” or “transformation” as referred to herein encompass the transfer of an exogenous polynucleotide into a host yeast cell, irrespective of the method used for transfer. Several methods of transformation in yeast cells have been developed. Some of the common methods used in transformation of yeast cells are lithium, electroporation, biolistic and use of the glass bead method. In summary, transformation involves three main steps; (1) preparing competent yeast cells; (2) transformation with the nucleic acid construct and (3) subsequent plating to select the transformants.

There is also provided the use of the genetically altered yeast described above in fermentation, and in particular, the use of the genetically altered yeast to increase the flavour of a fermented product, preferably when fermentation is carried out under high pressure, such as above 0.5 bar.

In another aspect of the invention, there is provided a method for identifying and selecting a yeast that is capable of increasing the flavour of a fermented product, the method comprising detecting in a yeast genome at least one polymorphism in at least one MDS3 gene and selecting said yeast. Preferably, the polymorphism is a T to C substitution at position 2171C of SEQ ID NO: 2, or a homologous position in a homologous sequence.

Suitable tests for assessing the presence of a polymorphism would be well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

In one embodiment, the method comprises
a) obtaining a nucleic acid sample from a yeast cell; and
b) carrying out nucleic acid amplification of one or more MDS3 alleles using one or more primer pairs.

In a further step, the method may comprise hybridising or mating the identified yeast as a first parent with a second yeast as a second parent, where the first parent differs from the second parent and then identifying the hybrid yeast.

In another aspect of the invention, there is provided a method of producing a genetically altered yeast of the invention, the method comprising introducing at least one mutation into at least one MDS3 gene. Preferably, the at least one mutation is introduced using mutagenesis or targeted genome editing, such as CRISPR as described above.

In one particular example, the method comprises introducing and expressing in the yeast a nucleic acid construct comprising a nucleic acid sequence operably linked to a regulatory sequence, wherein the nucleic acid sequence encodes a sgRNA as defined in SEQ ID NO: 5 or 6 or a variant thereof.

In another embodiment, there is provided a method for producing a hybrid yeast capable of increasing the flavour of a fermented product, the method comprising introducing at least one mutation into at least one MDS3 gene of a first haploid yeast using any of the methods described herein and hybridising the first haploid yeast cell or spore with a second haploid yeast cell or spore to produce a hybrid yeast. The method may further involve the step of identifying the resulting hybrid yeast. Hybrid yeasts are particularly industrially valuable as a result of the synergy (also called heterosis and hybrid vigour) that results from hybridising different parental strains and that means the hybrid can perform better than either parent in certain environmental conditions. For example, Garcia Sanchez and co-workers (2012) describe how crossing rare viable spores of a Saccharomyces pastorianus strain with those of an S. cerevisiae ale strain yielded hybrids with improved growth at higher temperatures and greater tolerance to higher ethanol concentrations.

In another aspect of the invention, there is provided a method for producing a hybrid yeast capable of increasing the flavour of a fermented product, the method comprising identifying and selecting a yeast that is capable of increasing the flavour of a fermented product, the method comprising detecting in a yeast genome screen at least one polymorphism in at least one MDS3 gene, preferably a T to C substitution at position 2171C of SEQ ID NO: 2, selecting said first yeast and hybridising a haploid of the first yeast cell or spore with a second haploid yeast cell or spore to produce a hybrid yeast. Also provided is a hybrid yeast obtained or obtainable by the method of the invention.

The yeast may be of any suitable species for use in fermentation. In one embodiment, the yeast is selected from the Saccharomyces sensu stricto species complex. This complex includes Saccharomyces cerevisiae, Saccharomyces pastorianus, Saccharomyces eubayanus, S. cerevisiae x kudriavzevii, S. uvarum, S. eubayanus x uvarum, S. bayanus, S. jurei and hybrids thereof. More preferably, the yeast is selected from Brewer's yeast (Saccharomyces sp.), such as Saccharomyces cerevisiae, Saccharomyces pastorianus, Saccharomyces eubayanus and hybrids thereof. The yeast may be a diploid or haploid; in some embodiments the yeast may be of higher ploidy, for example, tetraploid.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appIn cited documents”) and all documents cited or referenced in the appIn cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting examples.

### EXAMPLE 1: Methodology for Evaluation of Tolerance of the IAAc/Alc Ratio to High CO₂ Pressure in Small-Scale Fermentations

To mimic the high CO₂ pressure in large-scale industrial brewing fermentors, we designed a laboratory-scale fermentation system with increased CO₂ pressure applied from a gas bottle (FIG. 1A). The system consisted of a gas bottle with an initial pressure regulator up to 12 bar, connected via pressure resistant tubing to a more precise pressure control unit operating between 0 and 3 bar. From there, the gas flows to collectors in which 10 removable plugs can be fitted. These plugs are connected to 0.5L pressure-resistant fermentation bottles (up to 4 bar) through tubing with a filter for sterilization of the gas flow and a safety valve for release of excess pressure (>1 bar). The fermentation bottles are equipped with a sampling valve and placed on electromagnetic stirring plates for continuous stirring.

Isoamyl acetate is uniquely produced in yeast by the AATase enzymes, Atf1 and Atf2 that transform acetyl-coA and isoamyl alcohol into isoamyl acetate. High CO₂ pressure not only inhibits AATase activity, but also formation of the precursors isoamyl alcohol and other fusel alcohols. The ratio between isoamyl acetate and isoamyl alcohol (IAAc/Alc ratio) is therefore a more reliable readout for the AATase activity and its sensitivity to high CO₂ pressure. Using the lab-scale fermentation system with simulated CO₂ pressure, we first evaluated the effect of 0.50-1.50 bar CO₂ overpressure on the AATase activity of four representative brewing yeasts by quantification of the IAAc/Alc ratio at the end of the beer fermentations. As expected, the AATase activity was significantly inhibited by CO₂ overpressure of 0.50 and 0.65 bar in a strain specific manner, with the strongest inhibition observed for the JT28334 Frohberg lager yeast (FIGS. 1B and C). The final assay used for all subsequent pressurized fermentations applied 0.65 bar CO₂ overpressure, which caused a clear inhibition of AATase activity and only minimal inhibition of fermentation activity (FIG. 1D).

### EXAMPLE 2: Screening of Strains for Tolerance of the IAAc/Alc Ratio to High CO₂ Pressure

Due to the labour-intensive set-up of the CO₂ pressurized fermentations, we first made a preselection based on high isoamyl acetate production by screening 423 Saccharomyces spp. strains in semi-anaerobic non-pressurized fermentations with yeast extract-peptone medium adjusted to a free amino nitrogen concentration of 250 mg/L and containing 10% (w/v) glucose. From this prescreening, we selected the 100 strains with the highest isoamyl acetate production for evaluation in pressurized fermentation conditions. In addition, we included 100 strains previously reported to have a high ester profile. All these 200 strains showed good maltose fermentation capacity and were subjected to screening for superior CO₂ resilient AATase activity in fermentations with oxygenated malt extract medium under 0.65 bar CO₂ overpressure. When the fermentations were completed, we determined the metabolite profile with gas chromatography and derived the IAAc/Alc ratio as an indirect measure of the AATase activity. The results indicated that the IAAc/Alc ratio obtained for fermentations under inhibition by CO₂ was log-normally distributed with a tailing at the higher ratios (FIG. 2A). The 16 strains with an IAAc/Alc ratio of at least 0.06 were evaluated again in fermentations with and without CO₂ pressure to identify strains that maintained a high ratio in both conditions. Only three strains maintained an IAAc/Alc ratio over 0.08 both with and without CO₂ pressure, out of which the Sake strain Kyokai no. 1 (JT22329) showed the lowest production of isoamyl alcohol and minimal inhibition of AATase activity by high CO₂ pressure. This strain was therefore chosen for polygenic analysis of the underlying genetic elements.

### EXAMPLE 3: Selection of Superior Haploid Segregant from Superior Diploid Kyokai No. 1

Next, we deleted the two copies of the HO endonuclease gene in the diploid strain Kyokai no. 1 (JT22329) and sporulated the strain on minimal medium to obtain stable haploid spores. Although the parental strain was able to grow as well as ferment maltose, we observed segregation of the ability to ferment maltose in the haploid descendants. We therefore performed prescreening of 137 segregants for maltose fermentation in 24-well microtiter plates (FIG. 2B), and selected the 49 segregants that were able to fully complete the fermentation with malt extract. These were then evaluated in CO₂ pressurized fermentations, which revealed segregants that performed even better than the parental strain Kyokai no. 1 (JT22329). The superior segregant 63 (‘Seg.63’) was one of the best performing strains (FIG. 2C). We included in this evaluation several unrelated haploid strains for possible use as reference inferior strain in the polygenic analysis and finally selected the ER7A strain, a segregant from the Ethanol Red bioethanol production strain, because it displayed an intermediate IAAc/Alc ratio.

### EXAMPLE 4: Assembly of a Pool of Superior Segregants Obtained from the Hybrid Diploid Strain Seg.63/ER7A

We have mated Seg.63 with ER7A, sporulated the hybrid diploid Seg.63/ER7A and obtained haploid offspring with a range of IAAc/Alc ratios. Again, we observed a segregation in maltose fermentation capacity with 65% of the 428 isolated segregants showing high maltose fermentation capacity in microtiter plates, whereas the remainder showed incomplete fermentation of the maltose. The ploidy of the haploid segregants was confirmed by mating type PCR. Of the 279 maltose positive segregants, we screened 185 for strains displaying a high IAAc/Alc ratio under CO₂ pressurized conditions. The data were normalized against the IAAc/Alc ratio of the Kyokai no. 1 (JT22329) parental strain, which was included as a control in every batch of fermentations, to account for batch to batch variation. Segregation of the IAAc/Alc ratio in the progeny was normally distributed with a median of 91% of the IAAc/Alc ratio of the Kyokai no. 1 (JT22329) strain, and ranging between 75% of the IAAc/Alc ratio in the inferior ER7A strain and 115% of that in the superior Seg.63 strain (FIG. 2D). We selected 76 segregants with an IAAc/Alc ratio at least as high as that of Kyokai no. 1 (JT22329) for further evaluation and identified 28 segregants with an IAAc/Alc ratio at least 106% of that of Kyokai no. 1 (JT22329), which we used to compose the superior pool for polygenic analysis (FIG. 2E).

### EXAMPLE 5: QTL Mapping by Pooled-Segregant Whole-Genome Sequence Analysis

Genomic DNA was isolated from the pool of 28 maltose positive segregants with superior IAAc/Alc ratio and from two reference pools each composed of 33 segregants randomly selected with respect to the IAAc/Alc ratio, and either capable or incapable of complete maltose fermentation. The genomic DNA was subjected to Illumina whole-genome sequencing (BGI, Hong Kong). We performed QTL mapping by genome assembly of the sequence reads blasted against the S. cerevisiae S288c reference genome and plotting SNP variant frequency against SNP genomic position to create maps showing linkage disequilibrium along the genome. For the first pool of 28 maltose positive segregants selected for superior IAAc/Alc ratio, a QTLs was observed strongly linked to high IAAc/Alc ratio in CO₂ pressurized fermentations.

### EXAMPLE 6: Identification of MDS3 as the Causative Gene in QTL2

To further narrow down the linked area in QTL2, we performed bulk reciprocal hemizygosity analysis (RHA). Five kilobase pairs flanking each side of the predicted 1-LOD drop-off interval were included as a safeguard, resulting in a section of 116,059 to 165,091 bp in chromosome VII for RHA analysis. We divided this region into three blocks of 13.1, 17.5 and 16.9 kb, of which the regions from superior haploid Seg.63 or inferior haploid ER7A, respectively, were deleted separately in the Seg.63/ER7A hybrid. The IAAc/Alc ratio was evaluated in CO₂ pressurized fermentations with the reciprocally deleted hybrid strains. A significant drop in the IAAc/Alc ratio was observed for the strain with deletion of the block 1 fragment (116,059 to 129,161 bps) originating from the superior haploid strain, Seg.63 (FIG. 3A). We next performed single gene RHA for all genes in block 1 and identified the MDS3 gene as the major causative element in QTL2 (FIG. 3B). The MDS3 gene contained 10 missense mutations between the Seg.63 and ER7A alleles. The effect of deletion of the MDS3^{Seg 63} allele was striking, causing a drop with 71% of the IAAc/Alc ratio (student t-test, p=0.00008). This identified the MDS3 allele from Seg.63 as the major genetic element responsible for the difference between Seg.63 and ER7A in tolerance of the IAAc/Alc ratio to high CO₂ pressure. However, the deletion of the MDS3^{Seg 63} allele in the hybrid diploid strain also caused a growth defect resulting in slower fermentations that finished after only 6 days in comparison to 4 days for WT strains. This prompted us to investigate the MDS3 gene further in allele exchange experiments.

### EXAMPLE 7: MDS3 Allele Replacement in the Parental Strains Seg.63, ER7A and Seg.63/ER7A

To assess the importance of the MDS3 gene further, we deleted MDS3 in Seg.63, ER7A, and the Seg.63/ER7A hybrid with a NatMX selection marker flanked upstream and downstream by two Caenorhabditis elegans lir-2 (G2) protospacer sequences and performed allele exchange through Cas9-mediated cutting at the G2 sites while supplementing with PCR-amplified donor DNA. The causative character of MDS3 for conferring a superior IAAc/Alc ratio was confirmed by allele replacement in the inferior parent strain ER7A with the superior MDS3^{Seg 63} allele, which recovered 76% of the Seg.63 parental phenotype, corresponding to an increase with 71% of the IAAc/Alc ratio (FIG. 4A). Replacement of MDS3^{ER7A} in the Seg.63/ER7A hybrid diploid, to provide homozygosity with two MDS3^{Seg 63/Seg 63} alleles, did not increase the IAAc/Alc ratio further compared to the unmodified Seg.63/ER7A MDS3^{Seg 63/ER7A} strain (FIG. 4B). This supports that the superior MDS3^{Seg 63} allele is dominant, at least in the hybrid background. On the other hand, the IAAc/Alc ratio was strongly reduced in the haploid Seg.63 MDS3ER7A and hybrid diploid Seg.63/ER7A MDS3^{ER7A/ER7A} strains (FIGS. 4A and B). The Seg.63 and Seg.63/ER7A strains that only contained one or two MDS3ER7A alleles, respectively, showed a growth defect resulting in slower fermentations that were finished only after six days in comparison to the four days for the wild-type parental strains. There were no apparent effects of the MDS3^{Seg 63} allele on growth or fermentation capacity.

### EXAMPLE 8: Engineering of the Superior MDS3^{Seg 63} Allele in a Tetraploid Lager Strain

Given the industrial potential of the superior MDS3^{Seg 63} allele, we further investigated its applicability to engineer an S. pastorianus lager yeast, JT28325, using CRISPR/Cas9. The Frohberg type S. pastorianus lager yeast originates from a hybridization event between S. cerevisiae and S. eubayanus. It has an approximately tetraploid (aneuploid) genome. At the MDS3 locus, the Frohberg type strain contains four copies of the same allele, without any variants in the promoter, open reading frame or terminator region. It has 98.7% sequence similarity to MDS3 of the Seg.63 and ER7A S. cerevisiae strains. We used the CRISPR/Cas9 protocol described below, which allowed replacement of all four MDS3 alleles with the MDS3^{Seg 63} allele. Replacement of a single copy out of the four identical MDS3 copies in lager yeast strain JT28325 was performed by first knocking out a single gene and replacing the ORF using the G2 protospacer sequence, as described below. Both single and quadruple MDS3 replacement in the tetraploid lager yeast increased the IAAc/Alc ratio significantly, with a higher increase in case of replacement of all four alleles (61% and 145% increase, respectively; FIG. 5). This confirms that the MDS3^{Seg 63} allelic variant is dominant, and shows that it is also highly effective for improving the IAAc/Alc ratio in a completely unrelated and industrially highly relevant genetic background.

### EXAMPLE 9: Identification of the Causative Allelic Variant, MDS3^{T2171C} (Mds 3 ^{F724S})

We compared the ten MDS3 SNPs between Seg.63 and ER7A in the open reading frame with the sequence of the MDS3^{JT28325} allele present in the lager yeast JT28325. This revealed three unique missense mutations, C305T (T102M), T2171C (F724S), and A3229G (11077V) in the superior MDS3^{Seg 63} allele compared to the MDS3 alleles in the JT28325 strain. To identify which (combination) of the SNPs were causative, we performed genome editing of the MDS3 loci in the inferior ER7A strain using CRISPR/Cas9 and linear donor DNA constructs, containing all possible SNP combinations. Strikingly, the only causative SNP variant was T2171C, causing an amino acid change of phenylalanine to serine at position 724 in the Mds3 gene product. Any combination that included the T2171C variant yielded an IAAc/Alc ratio indistinguishable from that obtained after exchange of the entire MDS3 allele from Seg.63 (FIG. 6). Except for a slight increase in the ethyl octanoate level (“apple” descriptor), introduction of the T2171C variant did not affect the production of any other flavor compounds. Hence, we have identified a single SNP, T2171C, in the MDS3 gene, which can generate by itself a highly significant and specific enhancement of the IAAc/Alc ratio in fermentations under high CO₂ pressure.

### EXAMPLE 10: Materials and Methods

### Molecular Biology Methods

Yeast cells were transformed by electroporation or Gietz heat shock. Standard molecular biology protocols were used in this work.

### Cultivation Media

Yeast cells were grown at 30° C. in YPD medium [2% (w/V) glucose, 2% (w/v) peptone, 1% (w/v) yeast extract] with shaking at 200 rpm. For solid nutrient plates, 1.5% (w/V) Bacto agar was added. Escherichia coli cells (DH5, Invitrogen) were grown at 37° C. in Luria Broth (LB) medium containing 0.5% (w/v) yeast extract, 1% (w/v) Bacto tryptone, and 1% (w/v) sodium chloride (pH 7.5). For solid nutrient plates, 1.5% (w/v) Bacto agar was added. Selection of transformants was performed in the presence of 100 mg/L ampicillin.

### Screening in YP250-10% Glu Media

Flavor compound screening was performed in YP250 (0.27% yeast extract, Merck, 0.54% bacto peptone, Oxoid, to a total predicted nitrogen content of 250 mg/L and adjusted to pH 4.5 with concentrated hydrochloric acid) containing 10% (w/v) glucose.

The predicted nitrogen content was based on information of titratable nitrogen from the suppliers. The collection of strains was pre-cultured in 1 mL YP250-2% Glu, and fermentations were inoculated by volume with 0.5 mL culture in total volumes of 100 mL.

A Laboratory Scale Fermentation System with High CO₂ Pressure

The design of the system was done in collaboration with the suppliers of the equipment (Pneuvano, Wommelgem and KU Leuven Glasblazerij). The pressure resistant bottles, stirring rod, tubing with sterilization filter, safety valve and plug were autoclaved as a whole (assembled). After cooling, the safety release valves were set to 1 bar before addition of the medium. The rubber stops were penetrated by two glass tubes, one for CO₂-release and one for sampling, and the tubes were sealed with sterile cotton and a plastic tube with a clamp, respectively.

### Malt Extract Medium

Malt extract medium consisting of 166 g/L of malt extract (Brewferm spraymalt 8 EBC, Brouwland, Belgium) supplemented with 0.5 mg/L ZnSO₄, was autoclaved at 110° C. for 15 min. After autoclaving, the malt extract medium was cold settled overnight and filtered through a nylon filter (GE Healthcare) to remove insoluble precipitates. The final gravity of the malt extract medium was 15° P. Before fermentation, the medium was over-aerated by purging with pure oxygen supplied in a gas bottle to provide enough oxygen for the biosynthesis of unsaturated fatty acids. The oxygen level was approximately 20 mg/L, measured by an HQ30D dissolved oxygen meter with an LDO101 luminescent dissolved oxygen sensor (HACH).

### Inoculation of Beer Fermentations

The cells were inoculated from fresh YPD-plates into liquid YPD-medium in 3 mL volume (test tubes) and grown for 24 h at 30° C. with shaking at 200 rpm. 500 μL cell culture was subsequently transferred to 5 mL of malt extract (test tubes) and grown for 24 h at 30° C. with shaking at 200 rpm. The optical density was measured and 100 mL of malt extract was inoculated to an OD₆₀₀ of 1 in 300 mL shaking flasks. The cultures were grown for 2 days at 30° C. with shaking at 200 rpm.

### Assembly of Pools, Whole Genome Sequence Analysis and QTL Mapping

All strains were cultured separately in 4 mL cultures in 6-well plates for 2 days at 30° C. with shaking at 200 rpm in YPD containing 100 mg/L ampicillin and 10 mg/L doxycyclin The parent strains were grown in 32 mL cultures (8×4 mL in 6-well plates). The antibiotics were added to avoid any possibility of bacterial contamination. The mixing was done based on optical density (OD₆₀₀). Due to cell clumping the cultures were first sonicated to obtain a reliable value corresponding with the cell number obtained by colony counting. Genomic DNA was extracted and purified with the “Masterpure Yeast DNA purification kit” from Epicentre in order to obtain high quality genomic DNA.

The DNA was measured with the PicoGreen method (Quant-iT™ kit, Invitrogen). All the samples contained over 15 μg of DNA, which was subsequently sent to BGI (Hong Kong) for Illumina HiSeq2000 sequence analysis. Assembly and mapping were done with NGSEP (Next Generation Sequencing Eclipse Plugin) and linkage analysis was performed with MULTIPOOL.

### Reciprocal Hemizygosity Analysis

The deletion constructs for bulk RHA were amplified from the Euroscarf laboratory strain (BY4741) deletion collection according to the split marker method. This includes amplification and transformation of two PCR products per genomic target, each containing half of a KanMX marker cassette (split) with a 552 bp overlapping region between the two amplicons. The gene blocks were selected according to the availability of deletion strains and were chosen to have a size of 14 to 18 kb. To ensure efficient homologous recombination for the large deletions, we used long 0.4-1 kb flanking regions. For single gene RHA, 50 bp flanking regions were used.

### CRISPR/Cas9 Mediated Genome Editing

To perform direct allele replacement of the MDS3 gene in prototrophic yeast strains, we exchanged auxotrophic URA3 and TRP1 markers in the Cas9 guide RNA expression plasmids developed by DiCarlo et al. (46) with antibiotic selection markers. For cutting in two genetic loci, we constructed a plasmid by PCR amplification of fragments containing the first target guide RNA sequence fused to the trans-activating crRNA, tyrosine tRNA SUP4 terminator, and a snoRNA SNR52 promoter fused to the second guide RNA target sequence (gRNA1-SUP4t-SNR52p-gRNA2). Single gRNA oligo duplexed or 2×gRNA PCR fragments were inserted through Gibson cloning into the guide RNA expression plasmids for expression of two guide RNAs under the SNR52 promoter and SUP4/CYC1 terminators. The backbone p414-TEF1p-Cas9-CYC1t and p426-SNR52p-gRNA. CAN1.Y-SUP4t plasmids were a gift from George Church (Addgene plasmid #43802 and 43803).

Single allele replacement was done based on gene deletion by homologous recombination with a NatMX antibiotic marker flanked upstream and downstream by two Caenorhabditis elegans lir-2 (G2, 5′-GGATGAGAATCTGACAAAGG) (SEQ ID NO: 7) protospacer sequences (34). Deletion of the MDS3 ORF in ER7A and Seg.63 was carried with the primers MDS3-A1 FWD, TAAGGCAGACTCCGTGGAGTGTAAGAGAAGTTCAAAGCAAGGTTAGGCTTGTGGT CGGCTGGAGATCGG (SEQ ID NO: 8) and MDS3-A2 REV, GAATTTGAATTGTCCTGAGCTGACCTGGTCTGCCCGCTTCGATAAACTGCAGCCG TTATGGCGGGCATC (SEQ ID NO: 9), containing a 50 bp homology region up- and downstream of the ORF, and A1/A2 adaptors for amplification of the G2-NatMX-G2 cassette. The G2 gRNA targets were then subsequently targeted for efficient Cas9-mediated cutting, which allowed screening for loss of the antibiotic resistance marker, increasing the success rate for insertion of the alternative full-length MDS3 allele. Direct replacement of the MDS3 gene was performed using the following guide RNAs: 5′-GGGTAGCAGAAGCAAGCGGA (SEQ ID NO: 5), targeting the first (synonymous) mutation in the ORF; 5′-GATGTATAGCAGCATATTCT (SEQ ID NO: 6), targeting position 63 bp downstream of the ORF in the terminator.

CRISPR/Cas9 genome editing was commenced by first transforming yeast with a low copy number plasmid for stable and constitutive expression of the Cas9 endonuclease. Next, gRNA plasmids specific for the Seg.63 or ER7A MDS3 alleles or the G2 sequence were co-transformed with a PCR-amplified donor DNA (1 mg). MDS3 replacement with the marker cassette (targeting G2) was highly efficient in the haploid strains Seg.63 and ER7A (79% and 76%). The replacement efficiencies were also very high for other haploid strains (75-100%), but much less efficient for single replacement in the diploid Seg.63/ER7A hybrid (9-11%), and in an unrelated diploid brewing yeast (2%). The MDS3 replacement was unsuccessful in triploid and tetraploid strains, even after screening of 338 transformants. This is consistent with a previous report showing that CRISPR/Cas9 modification efficiency using the plasmids developed by DiCarlo et al. is lower for diploids than haploids. This is likely at least to some extent due to a strain specific shortfall of guide RNA expression observed in yeasts with higher ploidy. The reduced rates of successful replacement observed in the diploid, triploid and tetraploid strains were much lower than expected, probably due to the remaining MDS3 allele in the genome acting more efficient as donor DNA than the PCR amplified marker cassette. Because of the likely competition between PCR amplified donor template and chromosomal DNA for homologous repair, we tested whether transformation of the donor DNA on a 2-micron multi-copy plasmid (p426) could provide higher efficiency of correct repair. We therefore first transformed the lager yeast strain JT28325 with the multi-copy plasmid, containing the superior MDS3^{Seg 63} allele, and subsequently with plasmids expressing the Cas9 endonuclease and a guide RNA targeting G2, or two guide RNAs targeting the first mutation in the open reading frame and terminator. Transformation of the donor DNA plasmid prior to the guide RNA, with insertion of an antibiotic marker into the genome, increased the efficiency of single replacement from 0% to 40% in the tetraploid JT28325 lager yeast. Similarly, transformation of the donor DNA plasmid prior to the guide RNA, without insertion of an antibiotic marker into the genome, increased the efficiency of direct allele exchange from 0% to 8% for replacement of all four MDS3 alleles in the tetraploid JT28325 lager yeast.

### Construction of Variants of the MDS3^{Seg 63} Allele

To identify the causative variant in MDS3, we made linear donor DNA constructs with the high-fidelity NEB assembly mix, based on manufacturers instructions. We created four blocks covering the entire MDS3 ORF and flanked by 200 and 81 bp up- and downstream of the mds3: : G2-NatMX-G2 cassette. This was done by PCR amplification of fragments containing variants with the following primers: Block1 FWD, 5′-AAAGTCTATTTCAAGTTCACAG (SEQ ID NO: 10); Block1 REV (MDS3305C), 5′-TCTAGACATCAAGTCTAAGAAAAACGTCTC (SEQ ID NO: 11); Block1 REV (MDS3305T), 5′-TCTAGACATCAAGTCTAAGAAAAACATCTC (SEQ ID NO: 12); Block2 FWD: 5′-GTTTTTCTTAGACTTGATGTCTAGA (SEQ ID NO: 13); Block2 REV (MDS32171T), 5′-CGCTTTTTCCTTGAAAGGTACTCTGAAAA (SEQ ID NO: 14); Block2 REV (MDS32171C), 5′-CGCTTTTTCCTTGAAAGGTACTCTGGAAA (SEQ ID NO: 15); Block3 FWD, 5′-CAGAGTACCTTTCAAGGAAAAAGCG (SEQ ID NO: 16); Block3 REV (MDS33229A), 5′-AAGGTCTCCATCAACGAAGTACATATTAAG (SEQ ID NO: 17); Block3 REV (MDS33229G), 5′-AAGGTCTCCATCAACGAAGTACATACTAAG (SEQ ID NO: 18); Block4 FWD, 5′-TATGTACTTCGTTGATGGAGACCTT (SEQ ID NO: 19); Block4 REV: 5′-GGACGTAGCGGTCTATGG (SEQ ID NO: 20). Variants contained in primers are indicated in bold. The Gibson overlap was 25 bp with at least 50° C. annealing temperature. After fusion of the fragments by Gibson assembly for 1 h at 50° C., the products were purified to remove primer DNA and re-amplified with Block1 FWD and Block4 REV primers to create sufficient DNA (˜5 μg) for transformation into the ER7A haploid yeast.

### Headspace GC-FID Analysis

Headspace gas chromatography coupled with flame ionization detection (GC-FID) was used to measure flavor compounds at the end of the fermentation. Samples were collected and centrifuged at 3500 rpm for 5 min. Then, 5 ml of the supernatant was collected into 25-ml vials and analyzed using a gas chromatograph with a headspace sampler (Triplus RSH, Thermo Scientific). The headspace was equilibrated by shaking and incubation for 10 min at 60° C. and then injected into a polyethylene glycol column (Restek Stabilwax, 60m x 0.25 mm×0.25 μm).

Injection block and flame ionization detector temperatures were kept constant at 220 and 250° C., respectively. Oven temperature was kept at 40° C. for 2 min, then increased to 240° C. at a rate of 15° C./min. Helium was used as carrier gas at a flow rate of 2.0 mL/s. GC operating conditions were used as follows: injection volume 1 mL; split rate 1:25; split flow 50 mL/min.

### Mating Type Determination, Sporulation and Tetrad Dissection

Standard procedures were used for sporulation and tetrad dissection and for mating type determination by PCR with primers for MATa and MATa DNA at the MAT locus.

SEQUENCE LISTING
SEQ ID NO: 1 MDS3^{wild type} amino acid sequence
Amino acid
>MDS3 YGL197W SGDID: S000003165
MPLLQPSTCFCYPLKLPPLPLTSDSNEFDECARKRLTLDYRTGSAVTLTRSNIFVHGG
LTIPLNLPVVNSMQLQKELILFFAKEKNNGSSFRNLNEWISKETFFLDLMSRTWYRVKT
SFDQRTEELLKAESSSAKADNDTNEIRTDIKKGKSLESPLKERLFHSLCYLDGCLYIFG
GLTVSPQSGYELIATNELWKLDLNTKKWSLLSDDPQIARRFNHTMHVKNENNDNRDT
KLIIVGGLNNMDQPVKKIDIYNISQNCWQSETIPKQPMEITTNVNGIPLALSKDQNFSILV
ENNEANVPALAFYMRSDQIDEYLGKDSSKIKENSPIVALPLLSESQGIRMPSNPALPKK
LLNVPYELLAPTGDYFGFNIIIGGFHPNYQSSNFHCFIYDINSGKWSRVATACPDCDIN
KHRFWRVFVWKSHHQTILLGTKTDDYYSPSVQRFDHLSTFGLPLVNIFNKTIQLPHHKI
SASSLPIPIENFAKHKDTPLKKVSFTSSATSQFENYIRYIAPPLEMSSIQSVFPPYAMVL
GKDALEIYGKPLSDFEFITSEGDSIGIPCYLLRKRWGRYFDMLLSQSYTKVCADYETTD
TQSTLIKFSPHSSRNSSKAVRQEGRLSSSGSLDNYFEKNFPIFARTSVSEAQNTQPQV
ANADAKAPNTPSTSDEPSSSSSSDLYSTPHYQRNNDEEDDEDPVSPKPVSKSNSIYR

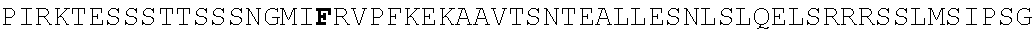

ELLRSSISEAEHQRRASHPLTSSPLFEDSGTPCGKQLQQLQQHTIQNPHNHLSPRRFS
RSARSSISYVSSSSDRRGNSISSRSTSDSFGTPPVLGVLNVPLPPQTREPNEPPPPCP
AMSTGSNTRRSNTLTDYMHSNKASPFSSRRSSHIGRRSSTPETENAFSATPRASLDG
QMLGKSLKEGSTSQYTQPRMNSFPKANETIQTPTSSNNEWSRQSVTSNTDSFDSLQ
SNFALELEPLLTPRSLYMPWPTSTVRAFAEFFYTGQVNSKWLLAPVALDLLVMAKIYEI
PLLYKLILEVLYSILAKKEESLSLICTSLMETFRTKTLNSYKGDEEKTNTYLTSNDNYQEL
LKLKVSLENIDNGYYDPDLLRKQSRAQSSSTQESSGSANGEKTATGAGSLETSSTNV
PTVFAGGPRDSHNSVGSIGFPNSMNIQGSRRSTSGFSPRVKMKSSLSKEIDPKTFYE
EYEPKEGKSFDDNDDQQTNIGSFNLHLFDMNYGSISSSSTNSISSSDLEEKEEQEQLQ
DLLEIEREDSAEILDARFRNKEDDKVTKDISNDKKRNYLPHEKNNLKAKEGKETRDVR
EEEEEFDFGLGMLSLNKIKREAKHVDKVDDSVDPLFKSSAFPQSPIRAYGSTTRTSSA
SGKPFRDNRSFNAFSVLTLENMASANALPPVDYVIKSIYRTTVLVNDIRLMVRCMDCIE
LSKNLRALKKKTMEDISKLKGISKPSP*
SEQ ID NO: 2 MDS3^{wild type} nucleic acid sequence
Coding DNA
>MDS3 YGL197W SGDID: S000003165
ATGCCTTTATTACAACCATCCACTTGCTTCTGCTACCCTTTGAAGCTTCCGCCATTA
CCACTGACTTCCGATTCCAACGAGTTTGATGAATGCGCCAGGAAAAGGTTAACGC
TGGACTATAGAACAGGTTCAGCGGTGACGCTAACAAGGTCTAATATATTTGTACAT
GGTGGTCTAACCATCCCGTTGAACCTGCCAGTTGTAAATTCTATGCAATTACAAAA
GGAGCTTATTCTCTTTTTTGCAAAGGAAAAAAACAACGGTAGTTCTTTTAGGAATTT
AAACGAGTGGATAAGTAAGGAGACGTTTTTCTTAGACTTGATGTCTAGAACCTGGT
ACAGAGTCAAGACCTCCTTTGACCAAAGGACAGAGGAATTATTAAAGGCAGAGAG
TTCTAGCGCCAAAGCAGATAATGACACAAACGAGATTAGAACGGACATTAAGAAA
GGTAAATCCCTTGAATCTCCGCTCAAGGAGAGGCTATTCCATTCCTTATGTTATTT
AGATGGCTGTTTATATATATTTGGTGGTCTCACAGTGTCTCCTCAGAGCGGATATG
AGTTGATTGCTACCAATGAGTTGTGGAAACTGGATTTAAACACGAAGAAGTGGTC
GCTTTTGAGTGATGACCCTCAAATAGCGAGAAGGTTCAACCATACCATGCATGTAA
AGAACGAAAATAACGATAATAGAGACACGAAATTAATAATTGTCGGAGGTCTTAAT
AACATGGATCAGCCGGTCAAGAAAATAGACATCTATAACATCTCACAGAATTGC
TGGCAATCCGAAACCATACCCAAACAACCGATGGAAATCACTACAAATGTCAATG
GCATACCATTAGCATTATCAAAGGATCAGAATTTTTCGATTTTAGTTGAAAATAATG
AGGCCAACGTTCCAGCGCTGGCATTCTATATGAGAAGTGATCAAATAGACGAATA
TCTCGGAAAGGATTCATCAAAGATAAAGGAGAATTCGCCTATAGTGGCGTTGCCG
CTATTGTCTGAATCGCAAGGTATTAGAATGCCCTCAAACCCGGCATTACCGAAAAA
ACTATTGAATGTTCCGTATGAGCTATTAGCACCGACAGGTGACTATTTTGGATTCA
ATATCATAATCGGTGGGTTTCATCCAAATTACCAATCCTCTAACTTTCATTGTTTTA
TATACGATATTAACTCCGGAAAGTGGTCACGAGTCGCTACCGCCTGCCCTGACTG
CGATATCAATAAACATCGATTCTGGAGGGTATTTGTTTGGAAATCGCATCATCAGA
CGATTTTGTTGGGTACCAAGACTGATGATTATTATTCACCCAGTGTACAAAGATTC
GACCATCTTTCCACTTTTGGATTACCGCTGGTAAATATTTTTAACAAGACGATCCAA
TTACCCCATCACAAGATCTCAGCTTCGTCTTTACCAATACCCATAGAAAATTTTGC
GAAGCATAAGGATACTCCATTAAAAAAGGTGAGCTTCACTTCATCAGCTACCTCCC
AATTTGAAAACTACATTAGATATATAGCTCCGCCCTTGGAAATGTCTTCTATCCAAT
CTGTGTTTCCACCGTATGCCATGGTTCTAGGTAAAGACGCTTTAGAGATTTACGGG
AAGCCTCTTTCTGATTTTGAATTTATTACTAGTGAAGGTGACTCCATTGGTATACCA
TGCTATTTGCTGAGGAAAAGATGGGGCCGTTATTTCGACATGCTTTTATCCCAGAG
TTATACAAAAGTTTGTGCTGATTATGAAACTACTGATACGCAATCTACACTAATAAA
ATTTTCACCACATTCATCTAGAAACAGTTCTAAAGCTGTGAGGCAAGAAGGTAGAC
TCTCTTCATCGGGTTCACTGGACAATTATTTCGAAAAAAACTTCCCAATCTTTGCGA
GAACAAGTGTTTCCGAGGCCCAGAACACACAACCTCAGGTAGCGAATGCTGATGC
GAAAGCTCCAAATACACCTAGTACGTCAGATGAACCAAGCTCTTCCTCCTCTTCCG
ATTTATACTCCACTCCTCATTACCAACGTAATAATGATGAAGAGGATGACGAGGAC
CCGGTTTCCCCGAAGCCTGTATCAAAATCAAACAGTATCTATAGACCCATTAGAAA

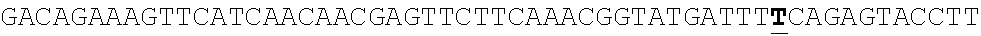

TCAAGGAAAAAGCGGCTGTAACGTCAAACACTGAAGCCCTCTTGGAGTCAAACCT
TTCACTTCAAGAATTGAGCCGAAGAAGATCATCACTTATGAGCATCCCGTCTGGTG
AACTTCTAAGGTCCTCTATATCTGAAGCGGAACATCAACGTCGTGCATCTCATCCA
CTCACTTCATCACCGCTCTTTGAAGATAGTGGAACCCCTTGCGGCAAACAACTGC
AACAACTGCAACAACATACTATACAAAATCCTCATAACCATTTGTCGCCCCGGAGG
TTTTCAAGAAGTGCAAGAAGTTCAATTTCCTACGTGAGTTCTTCTTCCGATAGACG
TGGAAATTCAATTTCTAGCAGGAGCACAAGCGATAGCTTTGGAACTCCACCAGTTT
TGGGCGTTTTGAATGTACCATTACCTCCTCAAACAAGAGAACCTAATGAACCACCT
CCACCATGTCCTGCAATGAGTACCGGCTCAAATACAAGGCGAAGCAACACCTTAA
CGGATTATATGCATTCTAACAAGGCTAGCCCGTTTTCTTCTCGTAGATCGTCGCAT
ATCGGTAGACGTTCTAGTACACCGGAAACGGAAAACGCATTCTCTGCAACACCAA
GAGCATCCTTAGATGGTCAAATGTTAGGAAAATCTTTGAAGGAAGGTTCTACATCA
CAGTACACACAGCCAAGGATGAACTCTTTCCCAAAGGCTAATGAAACCATACAAAC
ACCCACATCATCAAACAATGAATGGAGCCGTCAATCCGTTACTTCAAATACAGATA
GTTTTGACAGTTTGCAATCTAATTTTGCGTTAGAGTTGGAACCACTTTTAACACCGA
GGTCACTTTATATGCCTTGGCCAACCTCCACAGTTCGAGCATTTGCGGAATTTTTT
TACACTGGGCAAGTAAACAGTAAATGGCTTTTGGCACCAGTTGCTCTTGATTTATT
AGTAATGGCCAAGATTTATGAAATACCATTACTGTATAAATTGATCCTTGAAGTTTT
ATATTCGATTCTGGCTAAAAAAGAAGAAAGTTTATCCTTAATATGTACTTCGTTAAT
GGAGACCTTTCGCACCAAAACTCTGAACTCCTATAAAGGTGATGAAGAAAAAACA
AATACTTATTTGACTTCAAACGATAACTATCAGGAACTGTTGAAATTAAAAGTGTCG
CTGGAGAATATCGACAATGGGTATTATGACCCAGATTTGTTACGCAAACAGTCAAG
AGCACAGTCTTCAAGCACACAAGAAAGCAGTGGTAGTGCAAACGGCGAAAAAACT
GCAACTGGCGCTGGCTCTCTGGAGACTTCTTCAACCAATGTCCCTACAGTATTTG
CTGGCGGTCCCAGAGATAGCCACAATTCAGTAGGCTCCATTGGTTTTCCAAACAG
TATGAATATACAAGGCTCAAGAAGATCGACATCAGGATTTTCTCCACGCGTTAAGA
TGAAATCGAGCTTAAGCAAGGAAATAGATCCCAAAACTTTTTATGAAGAGTACGAA
CCAAAAGAGGGCAAAAGTTTTGATGATAACGATGATCAACAAACCAACATCGGAA
GTTTTAACCTTCATTTGTTTGATATGAATTATGGTTCCATCAGCAGCAGTAGCACTA
ACAGCATTAGTAGCAGTGATTTAGAAGAGAAAGAAGAACAAGAGCAACTTCAAGAT
TTATTGGAAATAGAAAGAGAAGATTCTGCTGAGATCTTAGACGCAAGATTCAGAAA
CAAAGAAGATGATAAAGTGACGAAGGATATATCAAATGACAAGAAACGCAATTACT
TACCGCATGAAAAAAATAACTTGAAAGCCAAGGAAGGTAAGGAAACTAGAGACGT
AAGGGAGGAGGAGGAAGAATTCGATTTTGGTTTGGGAATGCTTTCGCTCAATAAA
ATAAAAAGAGAAGCCAAGCATGTCGATAAGGTGGACGATTCTGTAGACCCTTTATT
TAAATCATCTGCTTTCCCTCAAAGTCCCATTCGAGCATACGGGTCTACCACAAGAA
CTAGTTCGGCTTCTGGAAAGCCATTCAGGGACAATCGTTCATTTAATGCATTTTCT
GTTTTGACATTAGAAAATATGGCGTCTGCAAATGCCCTACCTCCCGTTGATTATGT
CATAAAATCAATATACAGAACCACTGTGTTAGTGAACGATATCAGACTAATGGTTC
GTTGCATGGATTGCATTGAATTATCTAAAAATTTACGAGCGCTCAAGAAAAAAACT
ATGGAAGATATTTCGAAATTGAAAGGCATCTCTAAACCGTCACCATAG
SEQ ID NO: 3 MDS3F724S amino acid sequence
MPLLQPSTCFCYPLKLPPLPLTSDSNEFDECARKRLTLDYRTGSAVTLTRSNIFVHGG
LTIPLNLPVVNSMQLQKELILFFAKEKNNGSSFRNLNEWISKETFFLDLMSRTWYRVKT
SFDQRTEELLKAESSSAKADNDTNEIRTDIKKGKSLESPLKERLFHSLCYLDGCLYIFG
GLTVSPQSGYELIATNELWKLDLNTKKWSLLSDDPQIARRFNHTMHVKNENNDNRDT
KLIIVGGLNNMDQPVKKIDIYNISQNCWQSETIPKQPMEITTNVNGIPLALSKDQNFSILV
ENNEANVPALAFYMRSDQIDEYLGKDSSKIKENSPIVALPLLSESQGIRMPSNPALPKK
LLNVPYELLAPTGDYFGFNIIIGGFHPNYQSSNFHCFIYDINSGKWSRVATACPDCDIN
KHRFWRVFVWKSHHQTILLGTKTDDYYSPSVQRFDHLSTFGLPLVNIFNKTIQLPHHKI
SASSLPIPIENFAKHKDTPLKKVSFTSSATSQFENYIRYIAPPLEMSSIQSVFPPYAMVL
GKDALEIYGKPLSDFEFITSEGDSIGIPCYLLRKRWGRYFDMLLSQSYTKVCADYETTD
TQSTLIKFSPHSSRNSSKAVRQEGRLSSSGSLDNYFEKNFPIFARTSVSEAQNTQPQV
ANADAKAPNTPSTSDEPSSSSSSDLYSTPHYQRNNDEEDDEDPVSPKPVSKSNSIYR

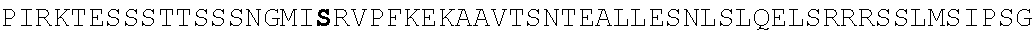

ELLRSSISEAEHQRRASHPLTSSPLFEDSGTPCGKQLQQLQQHTIQNPHNHLSPRRFS
RSARSSISYVSSSSDRRGNSISSRSTSDSFGTPPVLGVLNVPLPPQTREPNEPPPPCP
AMSTGSNTRRSNTLTDYMHSNKASPFSSRRSSHIGRRSSTPETENAFSATPRASLDG
QMLGKSLKEGSTSQYTQPRMNSFPKANETIQTPTSSNNEWSRQSVTSNTDSFDSLQ
SNFALELEPLLTPRSLYMPWPTSTVRAFAEFFYTGQVNSKWLLAPVALDLLVMAKIYEI
PLLYKLILEVLYSILAKKEESLSLICTSLMETFRTKTLNSYKGDEEKTNTYLTSNDNYQEL
LKLKVSLENIDNGYYDPDLLRKQSRAQSSSTQESSGSANGEKTATGAGSLETSSTNV
PTVFAGGPRDSHNSVGSIGFPNSMNIQGSRRSTSGFSPRVKMKSSLSKEIDPKTFYE
EYEPKEGKSFDDNDDQQTNIGSFNLHLFDMNYGSISSSSTNSISSSDLEEKEEQEQLQ
DLLEIEREDSAEILDARFRNKEDDKVTKDISNDKKRNYLPHEKNNLKAKEGKETRDVR
EEEEEFDFGLGMLSLNKIKREAKHVDKVDDSVDPLFKSSAFPQSPIRAYGSTTRTSSA
SGKPFRDNRSFNAFSVLTLENMASANALPPVDYVIKSIYRTTVLVNDIRLMVRCMDCIE
LSKNLRALKKKTMEDISKLKGISKPSP*
SEQ ID NO: 4 MDS3^{T2171} nucleic acid sequence
ATGCCTTTATTACAACCATCCACTTGCTTCTGCTACCCTTTGAAGCTTCCGCCATTA
CCACTGACTTCCGATTCCAACGAGTTTGATGAATGCGCCAGGAAAAGGTTAACGC
TGGACTATAGAACAGGTTCAGCGGTGACGCTAACAAGGTCTAATATATTTGTACAT
GGTGGTCTAACCATCCCGTTGAACCTGCCAGTTGTAAATTCTATGCAATTACAAAA
GGAGCTTATTCTCTTTTTTGCAAAGGAAAAAAACAACGGTAGTTCTTTTAGGAATTT
AAACGAGTGGATAAGTAAGGAGACGTTTTTCTTAGACTTGATGTCTAGAACCTGGT
ACAGAGTCAAGACCTCCTTTGACCAAAGGACAGAGGAATTATTAAAGGCAGAGAG
TTCTAGCGCCAAAGCAGATAATGACACAAACGAGATTAGAACGGACATTAAGAAA
GGTAAATCCCTTGAATCTCCGCTCAAGGAGAGGCTATTCCATTCCTTATGTTATTT
AGATGGCTGTTTATATATATTTGGTGGTCTCACAGTGTCTCCTCAGAGCGGATATG
AGTTGATTGCTACCAATGAGTTGTGGAAACTGGATTTAAACACGAAGAAGTGGTC
GCTTTTGAGTGATGACCCTCAAATAGCGAGAAGGTTCAACCATACCATGCATGTAA
AGAACGAAAATAACGATAATAGAGACACGAAATTAATAATTGTCGGAGGTCTTAAT
AACATGGATCAGCCGGTCAAGAAAATAGACATCTATAACATCTCACAGAATTGC
TGGCAATCCGAAACCATACCCAAACAACCGATGGAAATCACTACAAATGTCAATG
GCATACCATTAGCATTATCAAAGGATCAGAATTTTTCGATTTTAGTTGAAAATAATG
AGGCCAACGTTCCAGCGCTGGCATTCTATATGAGAAGTGATCAAATAGACGAATA
TCTCGGAAAGGATTCATCAAAGATAAAGGAGAATTCGCCTATAGTGGCGTTGCCG
CTATTGTCTGAATCGCAAGGTATTAGAATGCCCTCAAACCCGGCATTACCGAAAAA
ACTATTGAATGTTCCGTATGAGCTATTAGCACCGACAGGTGACTATTTTGGATTCA
ATATCATAATCGGTGGGTTTCATCCAAATTACCAATCCTCTAACTTTCATTGTTTTA
TATACGATATTAACTCCGGAAAGTGGTCACGAGTCGCTACCGCCTGCCCTGACTG
CGATATCAATAAACATCGATTCTGGAGGGTATTTGTTTGGAAATCGCATCATCAGA
CGATTTTGTTGGGTACCAAGACTGATGATTATTATTCACCCAGTGTACAAAGATTC
GACCATCTTTCCACTTTTGGATTACCGCTGGTAAATATTTTTAACAAGACGATCCAA
TTACCCCATCACAAGATCTCAGCTTCGTCTTTACCAATACCCATAGAAAATTTTGC
GAAGCATAAGGATACTCCATTAAAAAAGGTGAGCTTCACTTCATCAGCTACCTCCC
AATTTGAAAACTACATTAGATATATAGCTCCGCCCTTGGAAATGTCTTCTATCCAAT
CTGTGTTTCCACCGTATGCCATGGTTCTAGGTAAAGACGCTTTAGAGATTTACGGG
AAGCCTCTTTCTGATTTTGAATTTATTACTAGTGAAGGTGACTCCATTGGTATACCA
TGCTATTTGCTGAGGAAAAGATGGGGCCGTTATTTCGACATGCTTTTATCCCAGAG
TTATACAAAAGTTTGTGCTGATTATGAAACTACTGATACGCAATCTACACTAATAAA
ATTTTCACCACATTCATCTAGAAACAGTTCTAAAGCTGTGAGGCAAGAAGGTAGAC
TCTCTTCATCGGGTTCACTGGACAATTATTTCGAAAAAAACTTCCCAATCTTTGCGA
GAACAAGTGTTTCCGAGGCCCAGAACACACAACCTCAGGTAGCGAATGCTGATGC
GAAAGCTCCAAATACACCTAGTACGTCAGATGAACCAAGCTCTTCCTCCTCTTCCG
ATTTATACTCCACTCCTCATTACCAACGTAATAATGATGAAGAGGATGACGAGGAC
CCGGTTTCCCCGAAGCCTGTATCAAAATCAAACAGTATCTATAGACCCATTAGAAA

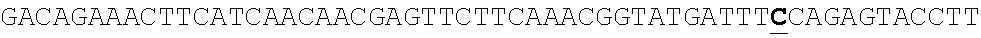

TCAAGGAAAAAGCGGCTGTAACGTCAAACACTGAAGCCCTCTTGGAGTCAAACCT
TTCACTTCAAGAATTGAGCCGAAGAAGATCATCACTTATGAGCATCCCGTCTGGTG
AACTTCTAAGGTCCTCTATATCTGAAGCGGAACATCAACGTCGTGCATCTCATCCA
CTCACTTCATCACCGCTCTTTGAAGATAGTGGAACCCCTTGCGGCAAACAACTGC
AACAACTGCAACAACATACTATACAAAATCCTCATAACCATTTGTCGCCCCGGAGG
TTTTCAAGAAGTGCAAGAAGTTCAATTTCCTACGTGAGTTCTTCTTCCGATAGACG
TGGAAATTCAATTTCTAGCAGGAGCACAAGCGATAGCTTTGGAACTCCACCAGTTT
TGGGCGTTTTGAATGTACCATTACCTCCTCAAACAAGAGAACCTAATGAACCACCT
CCACCATGTCCTGCAATGAGTACCGGCTCAAATACAAGGCGAAGCAACACCTTAA
CGGATTATATGCATTCTAACAAGGCTAGCCCGTTTTCTTCTCGTAGATCGTCGCAT
ATCGGTAGACGTTCTAGTACACCGGAAACGGAAAACGCATTCTCTGCAACACCAA
GAGCATCCTTAGATGGTCAAATGTTAGGAAAATCTTTGAAGGAAGGTTCTACATCA
CAGTACACACAGCCAAGGATGAACTCTTTCCCAAAGGCTAATGAAACCATACAAAC
ACCCACATCATCAAACAATGAATGGAGCCGTCAATCCGTTACTTCAAATACAGATA
GTTTTGACAGTTTGCAATCTAATTTTGCGTTAGAGTTGGAACCACTTTTAACACCGA
GGTCACTTTATATGCCTTGGCCAACCTCCACAGTTCGAGCATTTGCGGAATTTTTT
TACACTGGGCAAGTAAACAGTAAATGGCTTTTGGCACCAGTTGCTCTTGATTTATT
AGTAATGGCCAAGATTTATGAAATACCATTACTGTATAAATTGATCCTTGAAGTTTT
ATATTCGATTCTGGCTAAAAAAGAAGAAAGTTTATCCTTAATATGTACTTCGTTAAT
GGAGACCTTTCGCACCAAAACTCTGAACTCCTATAAAGGTGATGAAGAAAAAACA
AATACTTATTTGACTTCAAACGATAACTATCAGGAACTGTTGAAATTAAAAGTGTCG
CTGGAGAATATCGACAATGGGTATTATGACCCAGATTTGTTACGCAAACAGTCAAG
AGCACAGTCTTCAAGCACACAAGAAAGCAGTGGTAGTGCAAACGGCGAAAAAACT
GCAACTGGCGCTGGCTCTCTGGAGACTTCTTCAACCAATGTCCCTACAGTATTTG
CTGGCGGTCCCAGAGATAGCCACAATTCAGTAGGCTCCATTGGTTTTCCAAACAG
TATGAATATACAAGGCTCAAGAAGATCGACATCAGGATTTTCTCCACGCGTTAAGA
TGAAATCGAGCTTAAGCAAGGAAATAGATCCCAAAACTTTTTATGAAGAGTACGAA
CCAAAAGAGGGCAAAAGTTTTGATGATAACGATGATCAACAAACCAACATCGGAA
GTTTTAACCTTCATTTGTTTGATATGAATTATGGTTCCATCAGCAGCAGTAGCACTA
ACAGCATTAGTAGCAGTGATTTAGAAGAGAAAGAAGAACAAGAGCAACTTCAAGAT
TTATTGGAAATAGAAAGAGAAGATTCTGCTGAGATCTTAGACGCAAGATTCAGAAA
CAAAGAAGATGATAAAGTGACGAAGGATATATCAAATGACAAGAAACGCAATTACT
TACCGCATGAAAAAAATAACTTGAAAGCCAAGGAAGGTAAGGAAACTAGAGACGT
AAGGGAGGAGGAGGAAGAATTCGATTTTGGTTTGGGAATGCTTTCGCTCAATAAA
ATAAAAAGAGAAGCCAAGCATGTCGATAAGGTGGACGATTCTGTAGACCCTTTATT
TAAATCATCTGCTTTCCCTCAAAGTCCCATTCGAGCATACGGGTCTACCACAAGAA
CTAGTTCGGCTTCTGGAAAGCCATTCAGGGACAATCGTTCATTTAATGCATTTTCT
GTTTTGACATTAGAAAATATGGCGTCTGCAAATGCCCTACCTCCCGTTGATTATGT
CATAAAATCAATATACAGAACCACTGTGTTAGTGAACGATATCAGACTAATGGTTC
GTTGCATGGATTGCATTGAATTATCTAAAAATTTACGAGCGCTCAAGAAAAAAACT
ATGGAAGATATTTCGAAATTGAAAGGCATCTCTAAACCGTCACCATAG

1. A method of increasing the flavour of a fermented product, wherein the method comprises using a genetically altered yeast in fermentation and obtaining the fermented product, wherein the yeast is characterised by at least one mutation in at least one MDS3 gene.

2. The method of claim 1, wherein flavour of the fermented product is increased by increasing the ratio of isoamyl acetate (IAAc) to isoamyl alcohol (Alc).

3. The method of claim 1, wherein the carbon dioxide pressure during fermentation is above 0.5 bar.

4. The method of claim 1, wherein the MDS3 gene comprises a nucleic acid sequence that encodes a MDS3 polypeptide as defined in SEQ ID NO: 1 or a homologue or functional variant thereof.

5. The method of claim 1, wherein the mutation results in a substitution at position 724 of SEQ ID NO: 1 or a homologous position in a homologous sequence, preferably wherein the mutation is a F to S substitution.

6. (canceled)

7. The method of claim 1, wherein the yeast comprises a nucleic acid construct comprising a nucleic acid sequence encoding a MDS3 polypeptide as defined in SEQ ID NO: 3 or a fragment or variant thereof, preferably wherein the nucleic acid sequence comprises a sequence as defined in SEQ ID NO: 4 or a fragment or variant thereof, and optionally wherein the nucleic acid sequence is operably linked to a regulatory sequence.

8-9. (canceled)

10. The method of claim 1, wherein the yeast is selected from Saccharomyces cerevisiae, Saccharomyces pastorianus, Saccharomyces eubayanus, S. cerevisiae x kudriavzevii, S. uvarum, S. eubayanus x uvarum, S. bayanus, S. jurei and hybrids thereof.

11-14. (canceled)

15. The method of claim 11, wherein the fermented product is a fermented beverage.

16. (canceled)

17. A genetically altered yeast, wherein the yeast is characterised by at least one mutation in at least one MDS3 gene.

18. The genetically altered yeast of claim 17, wherein the mutation prevents or reduces the inhibition of flavour production by high carbon dioxide pressure when the yeast is used in fermentation.

19. The genetically altered yeast of claim 18, wherein the carbon dioxide pressure is above 0.5 bar.

20. The genetically altered yeast of claim 17, wherein the mutation increases the ratio of isoamyl acetate (IAAc) to isoamyl alcohol (Alc).

21. The genetically altered yeast claim 17, wherein the MDS3 gene comprises a nucleic acid sequence that encodes a MDS3 polypeptide as defined in SEQ ID NO: 1 or a homologue or functional variant thereof.

22. The genetically altered yeast claim 17, wherein the mutation results in a substitution at position 724 of SEQ ID NO: 1 or a homologous position in a homologous sequence, preferably wherein the mutation is a F to S substitution.

23. (canceled)

24. A genetically altered yeast, wherein the yeast comprises a nucleic acid construct comprising a nucleic acid sequence encoding a MDS3 polypeptide as defined in SEQ ID NO: 3 or a fragment or variant thereof.

25. The genetically altered yeast of claim 24, wherein the nucleic acid sequence comprises a sequence as defined in SEQ ID NO: 4 or a fragment or variant thereof.

26. The genetically altered yeast of claim 24, wherein the nucleic acid sequence is operably linked to a regulatory sequence.

27. The genetically altered yeast of claim 17, wherein the yeast is selected from the Saccharomyces sensu stricto species complex, optionally selected from Saccharomyces cerevisiae, Saccharomyces pastorianus, Saccharomyces eubavanus, S. cerevisiae x kudriavzevii, S. uvarum, S. eubavanus x uvarum, S. bayanus, S. jurei and hybrids thereof.

28-47. (canceled)

48. The genetically altered yeast of claim 24, wherein the yeast is selected from the Saccharomyces sensu stricto species complex, optionally selected from Saccharomyces cerevisiae, Saccharomyces pastorianus, Saccharomyces cubayanus, S. cerevisiae kudriavzevii, S. uvarum, S. cubayanus x uvarum, S. bayanus, S. jurei and hybrids thereof.
